(19) United States
(12) Patent Application Publication
(10) Pub. No.: US 2024/0218009 A1
(43) Pub. Date: Jul. 4, 2024

(54) NOVEL FORMS OF CYCLIC DINUCLEOTIDE COMPOUNDS

(71) Applicant: Merck Sharp & Dohme LLC, Rahway, NJ (US)

(72) Inventors: Stephanus Axnanda, Somerset, NJ (US); Andrew Patrick Jude Brunskill, Watchung, NJ (US); Erin N. Guidry, Cranford, NJ (US); Eric A. Kemp, Souderton, PA (US); Courtney K. Maguire, Watertown, MA (US); Mikhail Reibarkh, Cranford, NJ (US); Matthew S. Winston, Maplewood, NJ (US)

(73) Assignee: Merck Sharp & Dohme LLC, Rahway, NJ (US)

(21) Appl. No.: 18/553,774

(22) PCT Filed: Apr. 18, 2022
(86) PCT No.: PCT/US2022/025158

§ 371 Date: Oct. 3, 2023

Related U.S. Application Data
(60) Provisional application No. 63/177,503, filed on Apr. 21, 2021.

(51) Int. Cl. C07H 21/04

(52) U.S. Cl. CPC C07H 21/04

(57) ABSTRACT

Novel forms of 2-amino-9-[(2R,5R,7R,8S,10R,12aR, 14R,15S,15aR,16R)-14-(6-amino-9H-purin-9-yl)-15,16-difluoro-2,10-dihydroxy-2,10-disulfidooctahydro-12H-5,8-methanofuro[3,2-1][1,3,6,9,11,2,10]pentaoxadiphospha-cyclotetradecin-7-yl]-1,9-dihydro-6H-purin-6-one, which include adducts of 2-amino-9-[(2R,5R,7R,8S,10R,12aR, 14R,15S,15aR,16R)-14-(6-amino-9H-purin-9-yl)-15,16-difluoro-2,10-dihydroxy-2,10-disulfidooctahydro-12H-5,8-methanofuro[3,2-1][1,3,6,9,11,2,10]pentaoxadiphospha-cyclotetradecin-7-yl]-1,9-dihydro-6H-purin-6-one and L-histidine, may be useful as inductors of type I interferon production, specifically as STING active agents.

### FIELD OF THE INVENTION

The invention relates to novel forms comprising cyclic dinucleotide compounds that are STING (Stimulator of Interferon Genes) agonists that activate the STING pathway. The forms of the invention may be crystalline or amorphous.

### REFERENCE TO SEQUENCE LISTING SUBMITTED ELECTRONICALLY

The sequence listing of the present application is submitted electronically via EFS-Web as an ASCII-formatted sequence listing, with a file name of “25221WOPCT-SEQLIST-9FEB2022.txt”, creation date Feb. 9, 2022, and a size of 18,404 bytes. This sequence listing submitted via EFS-Web is part of the specification and is herein incorporated by reference in its entirety.

### BACKGROUND OF THE INVENTION

Compounds that induce type I interferon activity have great potential as anti-viral and anti-cancer agents (see T. R. Vargas et al., Rationale for STING-Targeted Cancer Immunotherapy, 75 Eur. J. Cancer 85-97 (2017); L. Corrales et al., Direct Activation of STING in the Tumor Microenvironment Leads to Potent and Systemic Tumor Regression and Immunity, 11 Cell Reports 1018-30 (2015); Glen N. Barber, STING: infection, inflammation and cancer, 15 Nat. Rev. Immunol. 760-770 (2015); E. Curran et al., STING Pathway Activation Stimulates Potent Immunity Against Myeloid Leukemia, 15 Cell Reports 2357-66 (2016). There is a growing body of data demonstrating that the cGAS-STING cGAS (cyclic GMP-AMP synthase-STING) cytosolic DNA sensory pathway has a significant capacity to induce type I interferons. Thus, the development of STING activating agents is rapidly taking an important place in today's anti-tumor therapy landscape.

Cyclic dinucleotide (CDN) compounds that are STING agonists for use in human subjects must be stored prior to use and transported to the point of administration. Reproducibly attaining a desired level of drug in a subject requires that the drug be stored in a formulation that maintains the potency of the drug. The need exists for stable forms of cyclic dinucleotide STING agonist compounds that can be formulated for pharmaceutical use, e.g., for treating cell proliferation disorders, such as cancers, and infectious diseases.

The cyclic dinucleotide STING agonist compound 2-amino-9-[(2R,5R,7R,8S,10R,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-1][1,3,6,9,11,2,10]pentaoxadiphospha-cyclotetradecin-7-yl]-1,9-dihydro-6H-purin-6-one and methods for making the same are illustrated in PCT International Patent Application No. PCT/US2016/046444, which published as PCT International Patent Application Publication No. WO2017/027646, and U.S. patent application Ser. No. 15/234,182, which published as U.S. Patent Application Publication No. US2017/0044206, which are incorporated herein by reference in their entirety, as Example 247. The present invention is directed to novel forms of 2-amino-9-[(2R,5R,7R,8S,10R,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-1][1,3,6,9,11,2,10]pentaoxadiphospha-cyclotetradecin-7-yl]-1,9-dihydro-6H-purin-6-one (Compound A).

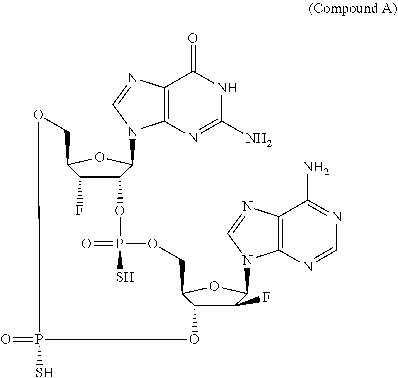 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1]

### SUMMARY OF THE INVENTION

This disclosure is directed to novel forms of 2-amino-9-[(2R,5R,7R,8S,10R,12aR, 14R,15S,15aR,16R)-14-(6-amino-9H-purin-9-yl)-15,16-difluoro-2,10-dihydroxy-2,10-disulfidooctahydro-12H-5,8-methanofuro[3,2-1][1,3,6,9,11,2,10]pentaoxadiphospha-cyclotetradecin-7-yl]-1,9-dihydro-6H-purin-6-one (Compound A). Certain forms have advantages, such as ease of processing, handling, or stability to stress. In particular, these forms exhibit improved physicochemical properties, such as stability to stress, rendering them particularly suitable for the manufacture of various pharmaceutical dosage forms. The disclosure also concerns pharmaceutical compositions containing the novel forms thereof, as well as methods for using them as STING agonists, particularly in the treatment of cell proliferation disorders, such as cancers. In certain embodiments, described herein are pharmaceutical compositions comprising a form of 2-amino-9-[(2R,5R,7R,8S,10R,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxadiphospha-cyclotetradecin-7-yl]-1,9-dihydro-6H-purin-6-one and a pharmaceutically acceptable carrier.

### BRIEF DESCRIPTION OF THE DRAWINGS

FIG. 1A depicts an XRPD diffraction pattern of a monosodium crystalline salt of Compound A in its initial phase.

FIG. 1B depicts an XRPD diffraction pattern of Compound A as a monosodium crystalline salt isolated from phosphate buffer.

FIG. 1C depicts an XRPD diffraction pattern of Form I of Compound A isolated from L-histidine buffer.

FIG. 2 depicts an XRPD diffraction pattern of Form I, showing characteristic reflections for Form I.

FIG. 3 depicts an ¹H-NMR spectrum of a washed dry cake of Form I, dissolved in dimethylsulfoxide, which shows the presence of L-histidine after a wash.

FIG. 4 depicts the aromatic region of ¹H-NMR spectra for Compound A in solution with varying concentrations of L-histidine.

### DETAILED DESCRIPTION OF THE INVENTION

This invention relates to forms of 2-amino-9-[(2R,5R,7R,8S,10R,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxadiphospha-cyclotetradecin-7-yl]-1,9-dihydro-6H-purin-6-one (Compound A):

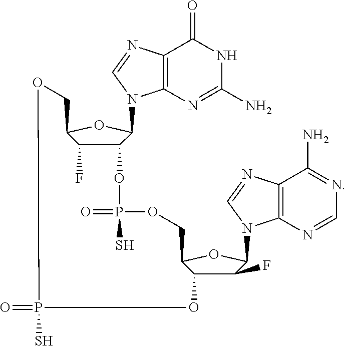 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1]

In particular, the invention relates to crystalline and amorphous forms of Compound A. Unless a specific form designation is given, the term “2-amino-9-[(2R,5R,7R,8S,10R,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxadiphospha-cyclotetradecin-7-yl]-1,9-dihydro-6H-purin-6-one” includes all forms described herein.

One embodiment of the forms described herein is an adduct of 2-amino-9-[(2R,5R,7R,8S,10R,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxa-diphospha-cyclotetradecin-7-yl]-1,9-dihydro-6H-purin-6-one and L-histidine (Form I). Form I is further described below.

A second embodiment of the present invention provides a particular drug substance that comprises at least one of the forms described herein. By “drug substance” is meant the active pharmaceutical ingredient. The amount of a form in the drug substance can be detected by physical methods such as X-ray powder diffraction, fluorine-19 magic-angle spinning (MAS) nuclear magnetic resonance spectroscopy, and carbon-13 cross-polarization magic-angle spinning (CPMAS) nuclear magnetic resonance spectroscopy.

In aspects of the second embodiment, the forms are characterized by having selected diagnostic peaks in an X-ray powder diffraction pattern (XRPD). In particular aspects of this embodiment, the forms are characterized by an X-ray powder diffraction (XRPD) containing at least 2 of the following 2θ values measured using CuKα radiation: about 5.32, about 6.77, about 10.41, about 11.11, about 11.38, about 12.57, about 12.83, about 13.87, about 14.51, about 14.88, about 15.92, about 16.28, about 17.48, about 18.95, about 19.16, about 19.79, about 20.59, about 21.15, about 21.76, about 22.30, about 22.80, about 23.01, about 23.19, about 23.54, about 24.17, about 26.64, about 26.90, about 27.50, about 28.33, about 28.86, about 29.89, about 30.19, about 30.85, about 31.46, about 31.78, about 32.22, about 32.89, about 33.62, about 34.50, about 35.30, about 36.07, about 37.18, about 37.80, and about 38.28° 20. In further aspects, the forms are characterized by an X-ray powder diffraction containing at least 2 of the following 2θ values measured using CuKα radiation: about 6.77, about 18.95, about 19.16, about 21.15, about 21.76, about 22.80, about 23.19, and about 24.17° 2θ. In still further aspects, the forms are characterized by an X-ray powder diffraction containing at least 2 of the following 2θ values measured using CuKα radiation: about 5.32, about 10.41, about 11.38, about 14.88, about 15.92, about 19.79, about 20.59, and about 23.01° 20. In even further aspects, the forms are characterized by an X-ray powder diffraction containing at least 2 of the following 2θ values measured using CuKα radiation: about 13.87, about 14.51, about 26.64, about 26.90, about 27.50, about 28.33, about 30.19, and about 33.62° 2θ. In additional further aspects, the forms are characterized by an X-ray powder diffraction containing at least 2 of the following 2θ values measured using CuKα radiation: about 11.11, about 12.57, about 12.83, about 16.28, about 17.48, about 22.30, about 23.54, about 28.86, about 29.89, about 30.19, about 31.46, about 31.78, about 32.22, about 32.89, about 34.50, about 35.30, about 36.07, about 37.18, about 37.80, and about 38.28° 20.

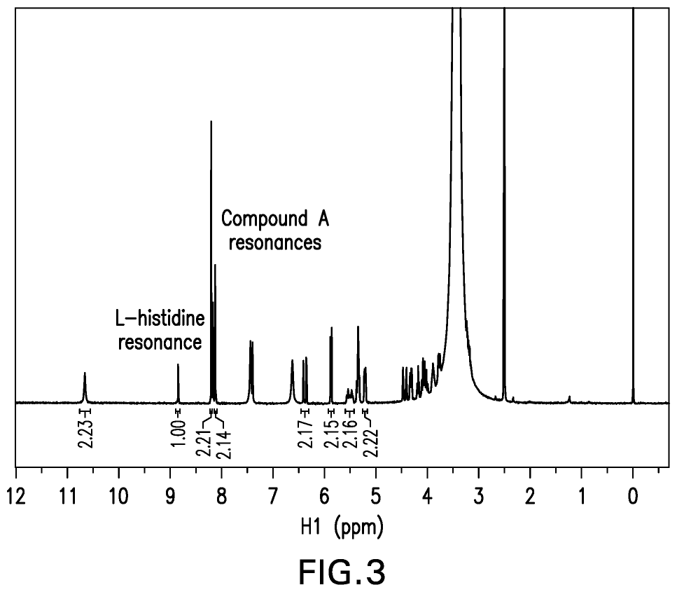

In additional aspects of the second embodiment, the forms are characterized by the proton nuclear magnetic resonance (¹H-NMR) spectra of FIG. 3.

Additional embodiments of the invention include pharmaceutical compositions comprising the forms described herein and a pharmaceutically acceptable carrier. The pharmaceutical compositions may be solid dosage forms for oral administration or sterile solutions for parenteral, intratumoral, intravenous, or intramuscular administration.

Further embodiments include the use of the forms described herein as an active ingredient in a medicament for inducing an immune response in a subject and the use of the forms described herein as an active ingredient in a medicament for inducing a STING-dependent type I interferon production in a subject. Further embodiments also include the use of the forms described herein as an active ingredient in a medicament for treatment of a cell proliferation disorder, which includes but is not limited to cancer.

Further embodiments include the use of the pharmaceutical compositions described herein as a medicament for inducing an immune response in a subject and for inducing a STING-dependent type I interferon production in a subject. Further embodiments also include the use of the pharmaceutical compositions described herein as an active ingredient in a medicament for treatment of a cell proliferation disorder, which includes but is not limited to cancer.

The forms of the present invention exhibit different chemical and physical properties as compared to the neutral form of Compound A as described in Example 247 of WO2017/027646 and US2017/0044206, which may provide pharmaceutical advantages. In particular, the novel forms, which have different equilibrium solubility values as compared to sodium salts of Compound A, enhanced chemical and physical stability of the forms constitute advantageous properties in the development of solid pharmaceutical dosage forms containing the pharmacologically active ingredient.

### Cell-Proliferation Disorders

The therapies disclosed herein are potentially useful in treating diseases or disorders including, but not limited to, cell-proliferation disorders. Cell-proliferation disorders include, but are not limited to, cancers, benign papillomatosis, gestational trophoblastic diseases, and benign neoplastic diseases, such as skin papilloma (warts) and genital papilloma. The terms “cancer”, “cancerous”, or “malignant” refer to or describe the physiological condition in mammals that is typically characterized by unregulated cell growth.

In specific embodiments, the disease or disorder to be treated is a cell-proliferation disorder. In certain embodiments, the cell-proliferation disorder is cancer. In particular embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary (i.e., cancers in which a metastasized cancer is found but the original cancer site is not known). In particular embodiments, the cancer is present in an adult patient; in additional embodiments, the cancer is present in a pediatric patient. In particular embodiments, the cancer is AIDS-related.

In specific embodiments, the cancer is selected from brain and spinal cancers. In particular embodiments, the brain and spinal cancer is selected from the group consisting of anaplastic astrocytomas, glioblastomas, astrocytomas, and estheosioneuroblastomas (also known as olfactory blastomas). In particular embodiments, the brain cancer is selected from the group consisting of astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astorcytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and suprantentorial primordial neuroectodermal tumors (sPNET).

In specific embodiments, the cancer is selected from cancers of the head and neck, including recurrent or metastatic head and neck squamous cell carcinoma (HNSCC), nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), and cancers of the eye or ocular cancers. In particular embodiments, the ocular cancer is selected from the group consisting of intraocular melanoma and retinoblastoma.

In specific embodiments, the cancer is selected from skin cancers. In particular embodiments, the skin cancer is selected from the group consisting of melanoma, squamous cell cancers, and basal cell cancers. In specific embodiments, the skin cancer is unresectable or metastatic melanoma.

In specific embodiments, the cancer is selected from cancers of the reproductive system. In particular embodiments, the cancer is selected from the group consisting of breast cancers, cervical cancers, vaginal cancers, ovarian cancers, endometrial cancers, prostate cancers, penile cancers, and testicular cancers. In specific instances of these embodiments, the cancer is a breast cancer selected from the group consisting of ductal carcinomas and phyllodes tumors. In specific instances of these embodiments, the breast cancer may be male breast cancer or female breast cancer. In more specific instances of these embodiments, the breast cancer is triple-negative breast cancer. In specific instances of these embodiments, the cancer is a cervical cancer selected from the group consisting of squamous cell carcinomas and adenocarcinomas. In specific instances of these embodiments, the cancer is an ovarian cancer selected from the group consisting of epithelial cancers.

In specific embodiments, the cancer is selected from cancers of the gastrointestinal system. In particular embodiments, the cancer is selected from the group consisting of esophageal cancers, gastric cancers (also known as stomach cancers), gastrointestinal carcinoid tumors, pancreatic cancers, gallbladder cancers, colorectal cancers, and anal cancer. In instances of these embodiments, the cancer is selected from the group consisting of esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, and anal squamous cell carcinomas.

In specific embodiments, the cancer is selected from liver and bile duct cancers. In particular embodiments, the cancer is liver cancer (also known as hepatocellular carcinoma). In particular embodiments, the cancer is bile duct cancer (also known as cholangiocarcinoma); in instances of these embodiments, the bile duct cancer is selected from the group consisting of intrahepatic cholangiocarcinoma and extrahepatic cholangiocarcinoma.

In specific embodiments, the cancer is selected from kidney and bladder cancers. In particular embodiments, the cancer is a kidney cancer selected from the group consisting of renal cell cancer, Wilms tumors, and transitional cell cancers. In particular embodiments, the cancer is a bladder cancer selected from the group consisting of urothelial carcinoma (a transitional cell carcinoma), squamous cell carcinomas, and adenocarcinomas.

In specific embodiments, the cancer is selected from bone cancers. In particular embodiments, the bone cancer is selected from the group consisting of osteosarcoma, malignant fibrous histiocytoma of bone, Ewing sarcoma, chordoma (cancer of the bone along the spine).

In specific embodiments, the cancer is selected from lung cancers. In particular embodiments, the lung cancer is selected from the group consisting of non-small cell lung cancer, small cell lung cancers, bronchial tumors, and pleuropulmonary blastomas.

In specific embodiments, the cancer is selected from malignant mesothelioma. In particular embodiments, the cancer is selected from the group consisting of epithelial mesothelioma and sarcomatoids.

In specific embodiments, the cancer is selected from sarcomas. In particular embodiments, the sarcoma is selected from the group consisting of central chondrosarcoma, central and periosteal chondroma, fibrosarcoma, clear cell sarcoma of tendon sheaths, and Kaposi's sarcoma.

In specific embodiments, the cancer is selected from glandular cancers. In particular embodiments, the cancer is selected from the group consisting of adrenocortical cancer (also known as adrenocortical carcinoma or adrenal cortical carcinoma), pheochromocytomas, paragangliomas, pituitary tumors, thymoma, and thymic carcinomas.

In specific embodiments, the cancer is selected from thyroid cancers. In particular embodiments, the thyroid cancer is selected from the group consisting of medullary thyroid carcinomas, papillary thyroid carcinomas, and follicular thyroid carcinomas.

In specific embodiments, the cancer is selected from germ cell tumors. In particular embodiments, the cancer is selected from the group consisting of malignant extracranial germ cell tumors and malignant extragonadal germ cell tumors. In specific instances of these embodiments, the malignant extragonadal germ cell tumors are selected from the group consisting of nonseminomas and seminomas.

In specific embodiments, the cancer is selected from heart tumors. In particular embodiments, the heart tumor is selected from the group consisting of malignant teratoma, lymphoma, rhabdomyosacroma, angiosarcoma, chondrosarcoma, infantile fibrosarcoma, and synovial sarcoma.

In specific embodiments, the cell-proliferation disorder is selected from benign papillomatosis, benign neoplastic diseases and gestational trophoblastic diseases. In particular embodiments, the benign neoplastic disease is selected from skin papilloma (warts) and genital papilloma. In particular embodiments, the gestational trophoblastic disease is selected from the group consisting of hydatidiform moles, and gestational trophoblastic neoplasia (e.g., invasive moles, choriocarcinomas, placental-site trophoblastic tumors, and epithelioid trophoblastic tumors).

In embodiments, the cell-proliferation disorder is a cancer that has metastasized, for example, liver metastases from colorectal cancer.

In embodiments, the cell-proliferation disorder is selected from the group consisting of solid tumors. In particular embodiments, the cell-proliferation disorder is selected from the group consisting of advanced or metastatic solid tumors. In more particular embodiments, the cell-proliferation disorder is selected from the group consisting of malignant melanoma, head and neck squamous cell carcinoma, and breast adenocarcinoma.

In particular embodiments, the cell-proliferation disorder is classified as stage III cancer or stage IV cancer. In instances of these embodiments, the cancer is not surgically resectable.

### Dosing & Formulations

The dosage regimen is selected in accordance with a variety of factors including type, species, age, weight, sex and medical condition of the patient; the severity of the condition to be treated; the route of administration; and the renal and hepatic function of the patient. An ordinarily skilled physician, veterinarian, or clinician can readily determine and prescribe the effective amount of the drug required to prevent, counter or arrest the progress of the condition.

The forms of the present invention may be formulated and administered in solid dosage forms, such as tablets, pills, capsules, powders, or granules, which are intended for oral administration. Formulation of the compositions according to the invention can conveniently be by methods known from the art, for example, as described in Remington's Pharmaceutical Sciences, 17th ed., 1995. Furthermore, the forms of the present invention may be formulated and administered in sterile solutions for parenteral, intratumoral, intravenous, or intramuscular administration.

In the methods of the present invention, the forms described herein may be formulated as the active pharmaceutical ingredient, and may be administered in admixture with suitable pharmaceutical diluents, excipients, or carriers (collectively referred to herein as ‘carrier’ materials) suitably selected with respect to the intended form of administration and consistent with conventional pharmaceutical practices, that is, oral tablets or sterile solutions for parenteral, intratumoral, intravenous, or intramuscular administration.

For instance, for oral administration in the form of a tablet or capsule, the form described herein can be combined with an oral, non-toxic, pharmaceutically acceptable, inert carrier (such as lactose, starch, sucrose, glucose, methyl cellulose, magnesium stearate, dicalcium phosphate, calcium sulfate, mannitol, sorbitol and the like). For parenteral, intratumoral, intravenous, or intramuscular administration in the form of a sterile solution, the form described herein may be combined with suitable excipients and non-toxic, pharmaceutically acceptable, inert carrier into a formulation that may be provided as a prepared dosage form in a pre-filled injection apparatus, as a lyophilized formulation to be reconstituted for injection, or as a sterile liquid to be diluted for injection.

### Abbreviations

(NH₄)₂HPO₄ Ammonium phosphate
(NH₄)₂SO₄ Ammonium sulfate
[HN(n-oct)₃]₂[SO₄] Tri-n-octylammonium hydrogen sulfate
¹³F-NMR ¹³F nuclear magnetic resonance spectroscopy
¹⁹F-NMR ¹⁹F nuclear magnetic resonance spectroscopy
¹H-NMR Proton nuclear magnetic resonance spectroscopy
2,4,6-collidine 2,4,6-Trimethylpyridine
2,6-lutidine 2,6-Dimethylpyridine
2′-FA (2R,3R,4R,5R)-5-(6-amino-9H-purin-9-yl)-4-fluoro-2-(hydroxymethyl)tetrahydrofuran-3-ol
2′-F-thio-AMP (O-{[(2R,3R,4S,5R)-5-(6-amino-9H-purin-9-yl)-4-fluoro-3-hydroxyoxolan-2-yl]methyl} O,O-dihydrogen phosphorothioate, also known as 2′-fluoro-thio-adenosine monophosphate
2′-F-thio-ATP 2′-fluoro-thio-adenosine triphosphate
2-Me-THF 2-Methyltetrahydrofuran
2-TBS 2-tert-butyl(((2R,3S)-3-((tert-butyldimethylsilyl)oxy)-2,3-dihydrofuran2-yl)methoxy)dimethylsilane
3′-FG 9-((2R,3S,4S,5R)-4-fluoro-3-hydroxy-5-(hydroxymethyl)tetrahydrofuran-2-yl)-2-(isobutylamino)-1,9-dihydro-6H-purin-6-one
3′-F-thio-GMP (2S,3R,4S,5R)-5-(2-amino-6-oxo-1,6-dihydro-9H-purin-9-yl)-3-fluoro-4-hydroxy-2-(mercaptomethyl)tetrahydrofuran-3-yl dihydrogen phosphate, also known as 3′-fluoro-thio-guanosine monophosphate
3′-F-thio-GTP 3′-fluoro-thio-guanosine triphosphate
³¹P-NMR ³¹P nuclear magnetic resonance spectroscopy
3-TBS N-((2S,3S,4R,5R)-4-((tert-butyldimethylsilyl)oxy)-5-(((tert-butyldimethylsilyl)oxy)methyl)-3-fluorotetrahydrofuran-2-yl)-N-(phenylsulfonyl) benzenesulfonamide
Ac Acetyl
AcP-Li/Li Dilithium acetylphosphate
AMP Adenosine monophosphate
aq Aqueous
ATP Adenosine 5′-triphosphate
BSA Bistrimethylsilyl acetamide
BSTFA Bistrimethylsilyl trifluoroacetamide, also referred to as trimethylsilyl 2,2,2-trifluoro-N-(trimethylsilyl)acetimidate
Bu Butyl
CD₂Cl₂ Deuterium-enriched dichloromethane
CD₃OD Deuterium-enriched methyl alcohol, deuterium-enriched methanol
CDCl₃ Deuterium-enriched trichloromethane
cGAS Cyclic GMP-AMP synthase
CHCl₃ Trichloromethane
CoSO₄ Cobalt sulfate
CPME Cyclopentylmethyl ether
d Doublet
D₂O Deuterium-enriched water
DBSI N,N-Dibenzenesulfonimide
DCM, CH₂Cl₂ Dichloromethane
DI water Deionized water
DMAP 4-Dimethylaminopyridine
DME, Glyme Dimethoxyethane
DMF N,N-Dimethylformamide
DMSO Dimethyl sulfoxide
eq., equiv. Equivalents
ES Electron spray
Et Ethyl
EtOAc Ethyl acetate
EtOH Ethyl alcohol, ethanol
g Grams
GMP Guanosine monophophate
GTP Guanosine 5′-triphosphate
h Hour
H₂O Water
HCl Hydrogen chloride
HDMS Hexamethyldisilazane
hept Heptet
HOAc, AcOH Acetic acid
HPLC High-performance liquid chromatography
Hz Hertz
IPA, i-PrOH Isopropyl alcohol
IPAc Isopropyl acetate
IPTG Isopropyl β-D-1-thiogalactopyranoside
J NMR Coupling constant
K₂CO₃ Potassium carbonate
KCl Potassium chloride
KF Potassium fluoride
kg Kilogram
KOH Potassium hydroxide
L, 1 Liter
LCMS Liquid chromatography-mass spectroscopy
m Multiplet
M Molarity, number of moles of solute per liter of solution
m/z Mass divided by charge number
Me Methyl
MeCN, ACN Acetonitrile
MeOH Methanol
mg Milligram
MgCl₂ Magnesium chloride
MHz Megahertz
min Minute(s)
ML Mother liquor
mL, ml Milliliter
mM Millimolar
mmol Millimole
mol mole
MS Mass spectrometry
Ms Methanesulfonyl
MTBE Methyl tert-butyl ether, methyl tertiary butyl ether
N Normality, number of mole equivalents per liter of solution
N₂ Nitrogen (gas)
Na₂SO₄ Sodium sulfate
NaCl Sodium chloride
NADPH Nicotinamide adenine dinucleotide phosphate
NaOH Sodium hydroxide
NFSI N-fluorobenzenesulfonimide
Ni Nickel
Ni-NTA Nickel nitrilotriacetic acid
nm Nanometer
NMI 1-Methylimidazole
NMP N-Methyl-2-pyrrolidone
NMR Nuclear magnetic resonance
OD Optical density
PBS Phosphate-buffered saline
PG Protecting group
PIV, Piv Pivalate, 2,2-Dimethylpropanoate
PSCl₃ Thiophosphoryl chloride
PTPI N,N-bis(diphenylthiophosphoryl)amide
Py Pyridine
q Quartet
RPM, rpm Revolutions per minute
RT, rt Room temperature, approximately 25° C.
s Singlet
sat Saturated
SIMS Secondary ion mass spectrometry
STAB Sodium triacetoxyborohydride
t Triplet
TBS tert-Butyldimethylsilyl
TBS-Cl tert-Butyldimethylsilyl chloride
TES 2-[(2-Hydroxy-1,1-bis(hydroxymethyl)ethyl)amino] ethanesulfonic acid, I-[Tris(hydroxymethyl)methyl]-2-aminoethanesulfonic acid, TES free acid
TFA Trifluoroacetic acid
TGDE, Tetraglyme Tetraethylene glycol dimethyl ether
THF Tetrahydrofuran
tol Toluene
T_{R} Retention time
Ts, OTs para-Toluenesulfonyl or tosyl
Ts-Cl para-Toluenesulfonyl chloride or tosyl chloride
UPLC Ultra Performance Liquid Chromatography
UV Ultraviolet wavelength or ultraviolet radiation
vol % Percent by volume or volume percent
Vol., vol., V Volumes
wt % Percent by weight or weight percent
ZnSO₄ Zinc sulfate
μg, ug Microgram
μL, μl, μL, μl Microliters
μM, uM Micromolar

### Preparation of Compound A

A method for preparing Compound A, as well as its diastereomers, is disclosed in WO2017/027646 and US2017/0044206, as Examples 244, 245, 246, and 247, 2-amino-9-[(5R,7R,8S,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxa-diphosphacyclo-tetradecin-7-yl]-1,9-dihydro-6H-purin-6-one (Diastereomers 1-3) and 2-amino-9-[(5R,7R,8S,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-1][1,3,6,9,11,2,10]pentaoxa-diphosphacyclo-tetradecin-7-yl]-1,9-dihydro-6H-purin-6-one (Diastereomer 4), respectfully.

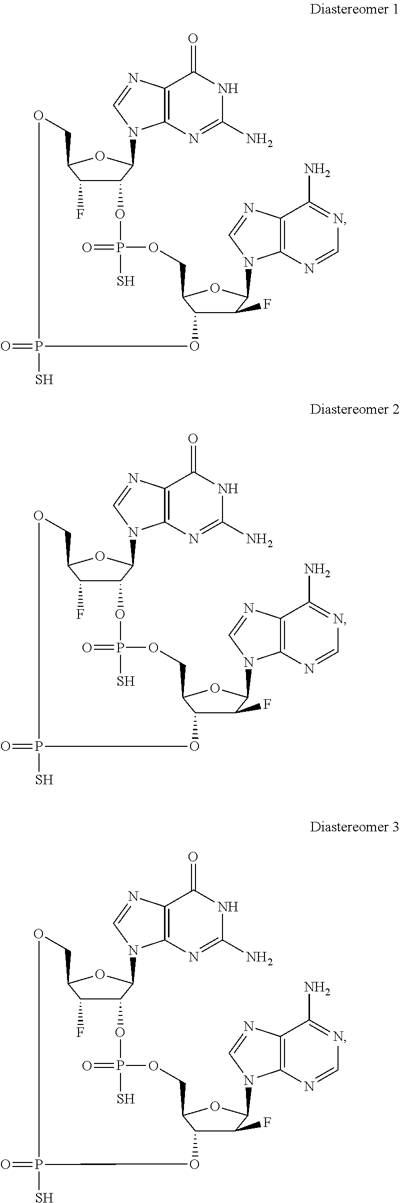 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1]

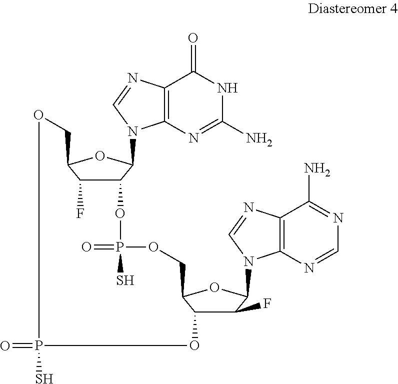 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1]

The compounds were prepared by the following process, as set forth in WO2017/027646 and US2017/0044206.

### Step 1: (2R,3S,4R,5R)-5-((((((2R,3R,4S,5R)-5-(6-benzamido-9H-purin-9-yl)-2-((bis(4-methoxyphenyl)(phenyl)methoxy)methyl)-4-fluorotetrahydrofuran-3-yl)oxy)(2-cyanoethoxy) phosphanyl)oxy)methyl)-4-fluoro-2-(2-isobutyramido-6-oxo-1,6-dihydro-9H-purin-9-yl) tetrahydrofuran-3-yl hydrogen phosphonate

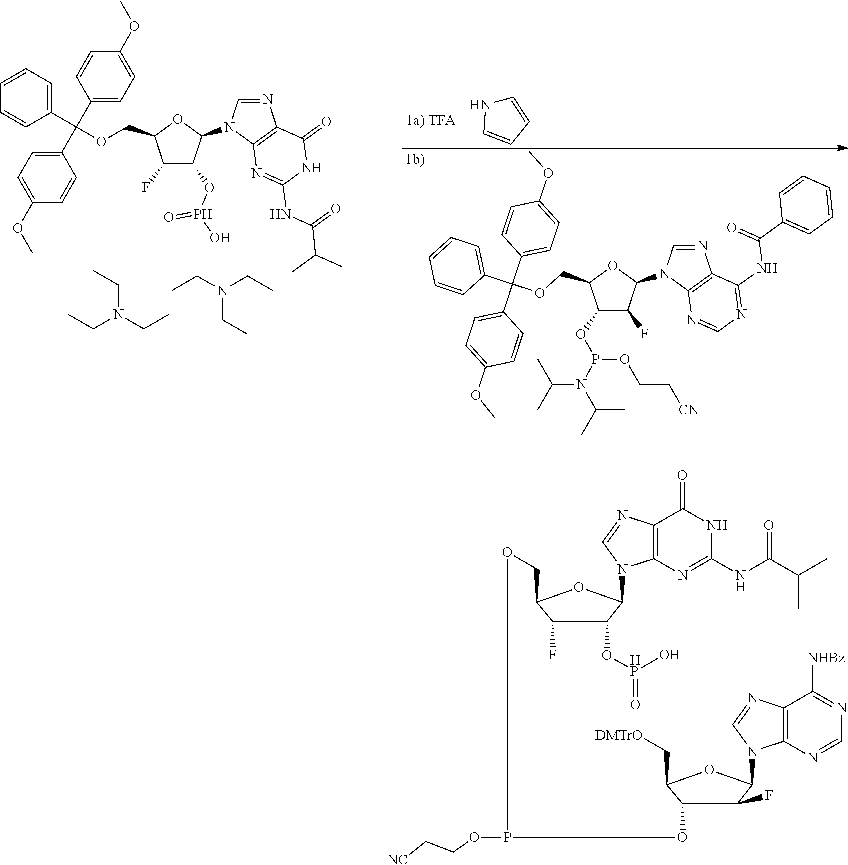

Pyrrole (0.087 mL, 1.2 mmol) was added to a solution of (2R,3S,4R,5R)-5-((bis(4-methoxyphenyl)(phenyl)methoxy)methyl)-4-fluoro-2-(2-isobutyramido-6-oxo-1,6-dihydro-9H-purin-9-yl)tetrahydrofuran-3-yl hydrogen phosphonate triethylamine salt (1:2) (0.34 g, 0.41 mmol) in acetonitrile (3.0 mL) under an argon atmosphere at 0° C. After 5 min, trifluoracetic acid (0.096 mL, 0.14 mmol) was added, and the reaction mixture was stirred at 0° C. for 30 min. Pyridine (0.13 mL, 1.7 mmol) was added drop wise at 0° C. The reaction mixture was then stirred for 10 min at 0° C. At that time, a mixture of (2R,3R,4S,5R)-5-(6-benzamido-9H-purin-9-yl)-2-((bis(4-methoxyphenyl)(phenyl)methoxy)methyl)-4-fluorotetrahydrofuran-3-yl-(2-cyanoethyl) diisopropylphosphoramidite (0.48 g, 0.55 mmol) in acetonitrile (3.0 mL) was added drop wise over 5 min to the reaction mixture under an argon atmosphere at 0° C. The reaction mixture was stirred at 0° C. for 20 min and immediately used in the next step without further manipulation.

### Step 2: (2R,3S,4R,5R)-5-((((((2R,3R,4S,5R)-5-(6-benzamido-9H-purin-9-yl)-4-fluoro-2-(hydroxymethyl)tetrahydrofuran-3-yl)oxy)(2-cyanoethoxy)phosphorothioyl)oxy)methyl)-4-fluoro-2-(2-isobutyramido-6-oxo-1,6-dihydro-9H-purin-9-yl)tetrahydrofuran-3-yl hydrogen phosphonate

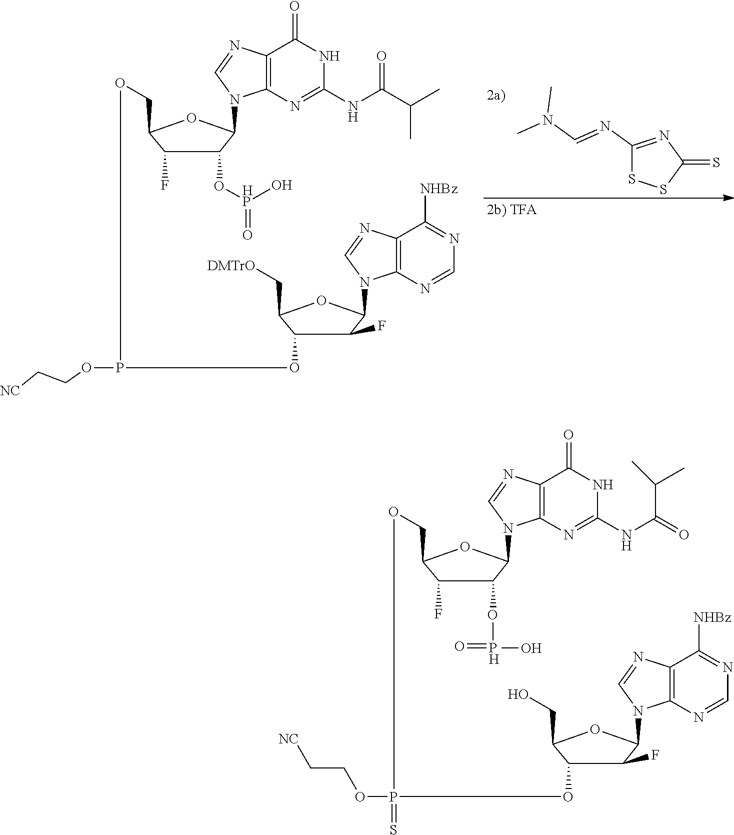

To the crude reaction mixture from Step 1 was added (E)-N,N-dimethyl-N′-(3-thioxo-3H-1,2,4-dithiazol-5-yl)formimidamide (0.10 g, 0.50 mmol) under an argon atmosphere at 0° C. The reaction mixture was stirred for 45 minutes at 0° C. At that time, 1-propanol (0.31 mL, 4.13 mmol) was added to the reaction mixture under an argon atmosphere at 0° C. The reaction mixture was then allowed to warm to ambient temperature and stirred for 10 min. TFA (0.32 mL, 4.1 mmol) was added to the reaction mixture, and the reaction mixture was stirred for 30 min at ambient temperature. Pyridine (0.37 mL, 4.6 mmol) was added at ambient temperature, and the reaction mixture was stirred for 10 min. The reaction mixture was concentrated under reduced pressure to approximately one-half volume. The mixture was then diluted with isopropyl acetate (20 mL) and stirred for 30 min at ambient temperature. The resulting suspension was filtered. The collected solids were dried overnight under high vacuum to afford (2R,3S,4R,5R)-5-((((((2R,3R,4S,5R)-5-(6-benzamido-9H-purin-9-yl)-4-fluoro-2-(hydroxymethyl)tetrahydrofuran-3-yl)oxy) (2-cyanoethoxy)phosphorothioyl) oxy)methyl)-4-fluoro-2-(2-isobutyramido-6-oxo-1,6-dihydro-9H-purin-9-yl)tetrahydrofuran-3-yl hydrogen phosphonate. LCMS (ES, m/z): 922 [M-H]⁻

### Step 3: 2-amino-9-[(5R,7R,8S,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxadiphosphacyclotetradecin-7-yl]-1,9-dihydro-6H-purin-6-one

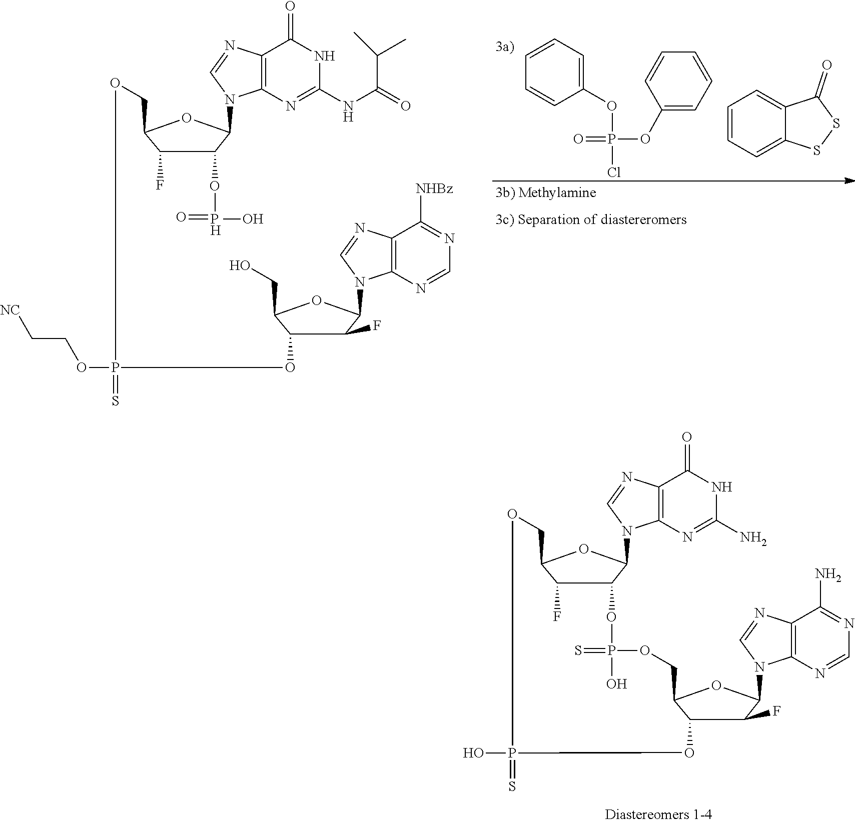

(2R,3S,4R,5R)-5-((((((2R,3R,4S,5R)-5-(6-benzamido-9H-purin-9-yl)-4-fluoro-2-(hydroxymethyl)tetrahydrofuran-3-yl)oxy)(2-cyanoethoxy)phosphorothioyl)oxy)methyl)-4-fluoro-2-(2-isobutyramido-6-oxo-1,6-dihydro-9H-purin-9-yl)tetrahydrofuran-3-yl hydrogen phosphonate (0.30 g, 0.33 mmol) was azeotroped with dry pyridine (2×10 mL) and then dried under high vacuum for 1 h. In a separate flask, diphenyl phosphorochloridate (0.34 mL, 1.6 mmol) was added to a mixture of acetonitrile (15 mL) and pyridine (1.0 mL). The resulting solution was then cooled to −20° C. To this mixture was added drop wise over a period of 5 min a mixture of (2R,3S,4R,5R)-5-((((((2R,3R,4S,5R)-5-(6-benzamido-9H-purin-9-yl)-4-fluoro-2-(hydroxy methyl)tetrahydrofuran-3-yl)oxy)(2-cyanoethoxy)phosphorothioyl)oxy)-methyl)-4-fluoro-2-(2-isobutyramido-6-oxo-1,6-dihydro-9H-purin-9-yl)tetrahydrofuran-3-yl hydrogen phosphonate (0.30 g, 0.33 mmol) in pyridine (4.0 mL) at −20° C. The reaction mixture was then stirred at −20° C. for 15 min post-addition. 3H-benzo[c][1,2]dithiol-3-one (0.066 g, 0.39 mmol) and water (0.12 mL, 6.5 mmol) were then added to the reaction mixture at −20° C. The reaction mixture was allowed to gradually warm to ambient temperature. The reaction mixture was stirred for 30 min at ambient temperature. The reaction mixture was then concentrated under reduced pressure to approximately one quarter volume. The reaction mixture was cooled to 0° C., and methylamine (33% in ethanol) (2.63 mL, 24 mmol) was added drop wise. After the addition was complete, the reaction mixture was allowed to warm to ambient temperature. The reaction mixture was stirred at ambient temperature for 18 h. The reaction mixture was concentrated under reduced pressure to afford the crude product residue. The crude product residue was azeotroped (3×30 mL ethanol) to afford the crude product. This material was dissolved in water (5 mL) and acetonitrile (1 mL). The resulting mixture was purified by mass-directed reverse phase HPLC (Waters Sunfire 19×250 mm, UV 215/254 nm, fraction trigger by SIMS negative MS monitoring mass 709; mobile phase=100 mM triethylammonium acetate in water/acetonitrile gradient, 2-30% acetonitrile over 40 min) to afford the 4 diastereomers of 2-amino-9-[(5R,7R,8S,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-l][1,3,6,9,11,2,10]pentaoxa-diphosphacyclotetradecin-7-yl]-1,9-dihydro-6H-purin-6-one.

Diastereomer 1: 2-amino-9-[(5R,7R,8S,12aR,14R,15S,15aR,16R)-14-(6-amino-9H-purin-9-yl)-15,16-difluoro-2,10-dihydroxy-2,10-disulfidooctahydro-12H-5,8-methanofuro [3,2-l][1,3,6,9,11,2,10]pentaoxadiphosphacyclotetradecin-7-yl]-1,9-dihydro-6H-purin-6-one: T_{R}=17.7 min. LCMS (ES, m/z): 709 [M-H]⁻.

Diastereomer 2: 2-amino-9-[(5R,7R,8S,12aR,14R,15S,15aR,16R)-14-(6-amino-9H-purin-9-yl)-15,16-difluoro-2,10-dihydroxy-2,10-disulfidooctahydro-12H-5,8-methanofuro [3,2-1][1,3,6,9,11,2,10]pentaoxadiphosphacyclotetradecin-7-yl]-1,9-dihydro-6H-purin-6-one: T_{R}=21.9 min. LCMS (ES, m/z): 709 [M-H]⁻. ¹H NMR (500 MHz, DMSO-d₆) δ 8.32 (s, 1H), 8.21-8.09 (m, 2H), 7.46-7.29 (m, 2H), 6.59-6.43 (m, 2H), 6.40-6.29 (m, 1H), 5.88 (d, J=8.8 Hz, 1H), 5.49-5.19 (m, 4H), 4.45-4.32 (m, 2H), 4.10-3.93 (m, 2H), 3.94-3.82 (m, 1H), 3.80-3.68 (m, 1H).

Diastereomer 3: 2-amino-9-[(5R,7R,8S,12aR,14R,15S,15aR,16R)-14-(6-amino-9H-purin-9-yl)-15,16-difluoro-2,10-dihydroxy-2,10-disulfidooctahydro-12H-5,8-methanofuro [3,2-1][1,3,6,9,11,2,10]pentaoxadiphosphacyclotetradecin-7-yl]-1,9-dihydro-6H-purin-6-one: T_{R}=23.8 min. LCMS (ES, m/z): 709 [M-H]⁻. ¹H NMR (500 MHz, DMSO-d₆) δ 8.18-8.08 (m, 3H), 7.41-7.33 (m, 2H), 6.59-6.47 (m, 2H), 6.37-6.27 (m, 1H), 5.84 (d, J=8.7 Hz, 1H), 5.52-5.26 (m, 2H), 5.21-5.11 (m, 1H), 4.46-4.35 (m, 2H), 4.19-4.02 (m, 2H), 3.83-3.65 (m, 2H).

Diastereomer 4, Compound A: 2-amino-9-[(2R,5R,7R,8S,10R,12aR,14R,15S,15aR,16R)-14-(6-amino-9H-purin-9-yl)-15,16-difluoro-2,10-dihydroxy-2,10-disulfidoocta-hydro-12H-5,8-methanofuro[3,2-l][1,3,6,9,11,2,10]pentaoxadiphosphacyclotetradecin-7-yl]-1,9-dihydro-6H-purin-6-one: T_{R}=26.4 min. LCMS (ES, m/z): 709 [M-H]⁻. ¹H NMR (500 MHz, DMSO-d₆) δ 8.19-8.07 (m, 3H), 7.41-7.32 (m, 2H), 6.70-6.50 (m, 2H), 6.40-6.29 (m, 1H), 5.85 (d, J=8.7 Hz, 1H), 5.33-5.25 (m, 2H), 5.23-5.12 (m, 1H), 4.48-4.35 (m, 1H), 4.33-4.24 (m, 1H), 4.09-3.93 (m, 2H), 3.92-3.81 (m, 1H), 3.83-3.70 (m, 1H).

Compound A also may be prepared from (O-{[(2R,3R,4S,5R)-5-(6-amino-9H-purin-9-yl)-4-fluoro-3-hydroxyoxolan-2-yl]methyl} O,O-dihydrogen phosphorothioate (also known as 2′-fluoro-thio-adenosine monophosphate or 2′-F-thio-AMP) and (2S,3R,4S,5R)-5-(2-amino-6-oxo-1,6-dihydro-9H-purin-9-yl)-3-fluoro-4-hydroxy-2-(mercaptomethyl) tetrahydrofuran-3-yl dihydrogen phosphate (also known as 3′-fluoro-thio-guanosine monophosphate or 3′-F-thio-GMP) as starting materials, as disclosed in U.S. Provisional Patent Application No. 63/170,003, filed Apr. 2, 2021. (O-{[(2R,3R,4S,5R)-5-(6-amino-9H-purin-9-yl)-4-fluoro-3-hydroxyoxolan-2-yl]methyl} O,O-dihydrogen phosphorothioate (also known as 2′-fluoro-thio-adenosine monophosphate or 2′-F-thio-AMP) may be prepared from processes including those disclosed in U.S. Provisional Patent Application No. 63/080,381, filed on Sep. 18, 2020. (2S,3R,4S,5R)-5-(2-amino-6-oxo-1,6-dihydro-9H-purin-9-yl)-3-fluoro-4-hydroxy-2-(mercaptomethyl) tetrahydrofuran-3-yl dihydrogen phosphate (also known as 3′-fluoro-thio-guanosine monophosphate or 3′-F-thio-GMP) may be prepared from processes including those disclosed in U.S. Provisional Patent Application No. 63/028,741, filed on May 22, 2020.

### PREPARATORY EXAMPLES

### Preparatory Example 1: Synthesis of (trisodium O-{[(2R,3S,4S,5R)-5-(2-amino-6-oxido-9H-purin-9-yl)-3-fluoro-4-hydroxyoxolan-2yl]methyl} phosphorothioate hydrate (1:6))

### Step 1: Synthesis of (2R,3R,4R,5R)-2-(2-amino-6-oxo-1,6-dihydro-9H-purin-9-yl)-5-(((tert-butyldimethylsilyl)oxy)methyl)tetrahydrofuran-3,4-diylbis(4-methylbenzenesulfonate)

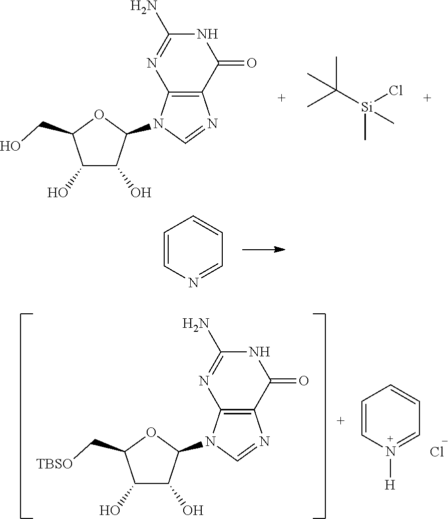

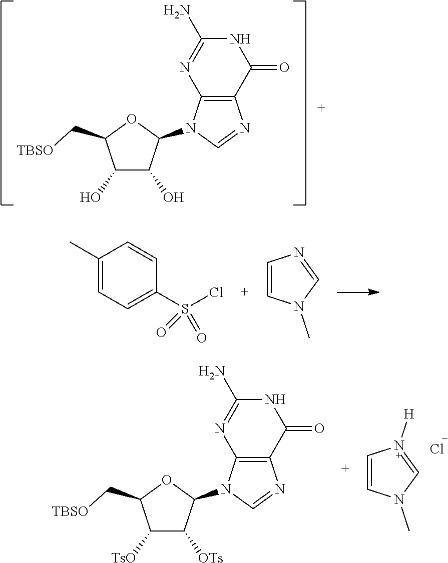

NMP (3.5 vol.) was added into a reaction vessel, and the temperature was adjusted to 48° C. to 52° C. Guanosine (800 g, 2824 mmol) was added. The reaction mixture was stirred for 30 min. to 1 h, and the temperature was adjusted to 8° C. to 12° C. TBS-Cl (575 g, 3815 mmol) (dissolved in 2 vol. NMP) was added into the reaction mixture (total NMP 5.5 vol.), and the reaction mixture was maintained at 8° C. to 12° C. Py (670 g, 8470 mmol) was added to the reaction mixture, which was maintained at 8° C. to 12° C. and stirred for 3 to 4 h. The temperature was adjusted to −20° C. to −10° C. and stir for 8 to 15 h, after which the temperature was adjusted to −5° C. to 5° C.

NMI (2319 g, 28240 mmol) was added to the reaction mixture, which was kept at −5° C. to 5° C. To the reaction mixture, 2.1 eq. Ts-Cl (1131 g dissolved in 3 vol. 2-Me-THF) was added, and the reaction mixture was stirred at −5° C. to 5° C. for 4 to 8 h. Then, 0.7 eq. Ts-Cl (377 g, dissolved in 1 vol. 2-Me-THF) was added. The reaction mixture was stirred for 12 to 14 h at −5° C. to 5° C. Ts-Cl (0.16 eq, 86 g dissolved in 160 mL 2-Me-THF) was added to the reaction mixture, which was stirred for 3 to 5 h. MeOH (5.5 vol.) was added to the reaction mixture at 15° C. to 25° C., followed by water (8 vol.). The reaction mixture was stirred at this temperature for 12 to 15 h. The reaction mixture was then filtered and rinsed with 2 vol. MeOH/water (1:3). The reaction product was dried under 45° C. for 70 h in two parts.

### Step 2: Synthesis of (2R,3R,4R,5R)-2-(((tert-butyldimethylsilyl)oxy)methyl)-5-(2-isobutyramido-6-oxo-1,6-dihydro-9H-purin-9-yl)tetrahydrofuran-3,4-diyl bis(4-methylbenzenesulfonate)

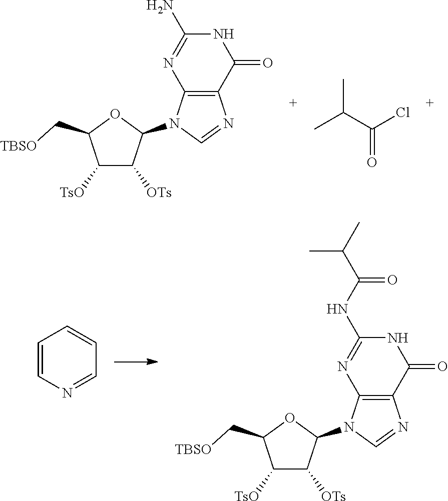

The bis-tosylate (1096.00 g (1185.50 g×92.45%)) was charged into a reaction vessel. MeCN (3.3 L, 3 vol.) and Py (510.52 g, 4.2 eq.) then were charged into the reaction vessel. The reaction mixture was cooled to −15° C. to −5° C. (slurry). Isobutyryl chloride (397.51 g, 2.4 eq.) was added by dropwise to the reaction mixture under −5° C. (slurry). The reaction mixture was stirred at −15° C. to −5° C. for 18 h. Isopropyl acetate (6 L, 5 vol.) was charged into the reaction mixture, and 15% K₂C03 liquor (6 kg) was added by dropwise into the reaction mixture under −5° C. The reaction mixture was stirred at −15° C. to −5° C. for 30 min. The reaction mixture was then warmed to 20° C. to 30° C. and stirred for 10 to 30 min.

The reaction mixture was separated, and the aqueous layer was removed. The organic layer was concentrated to 3-4 vol. at 30° C. IPAc (6 L, 5-6 vol.) was charged into the concentrated organic layer, which was then stirred at 25° C. to 30° C. for 30 min. The organic layer was then further concentrated until it reached 5-6 vol. under 30° C. An additional IPAc (2 L, 2-3 vol.) was charged into the concentrated organic layer, and it was stirred at 25° C. to 30° C. for 30 min. The reaction mixture was cooled to 15° C. to 25° C. 3 L (3 vol.) n-heptane was added drop-wise at 15° C. to 25° C. for 6 h, then the reaction mixture was stirred for 10 h 25° C. to 30° C. 3 L n-heptane was added drop-wise at 15° C. to 25° C. for 6 h, and the reaction mixture was stirred at 25° C. to 30° C. for 10 h. The suspension was filtered, and the filter cake was washed with 2 L mixture solution (IPAc/n-heptane=1 L/1 L) to give the product, which was dried in oven under 35° C. by reduce for 24 h.

¹H NMR (500 MHz, DMSO-d₆) δ 11.97 (s, 1H), 11.50 (s, 1H), 7.89 (d, J=8.3 Hz, 2H), 7.86 (s, 1H), 7.55 (d, J=8.2 Hz, 2H), 7.38 (d, J=8.2 Hz, 2H), 7.07 (d, J=8.2 Hz, 2H), 6.00 (d, J=7.9 Hz, 1H), 5.58 (dd, J=7.8, 5.4 Hz, 1H), 5.05 (d, J=5.3 Hz, 1H), 4.27 (t, J=4.5 Hz, 1H), 3.85 (dd, J=12.2, 4.1 Hz, 1H), 3.70-3.66 (m, 1H), 2.76 (septet, J=6.8 Hz, 1H), 2.46 (s, 3H), 2.26 (s, 3H), 1.18 (t, J=7.2 Hz, 6H), 0.87 (s, 9H), 0.06 (s, 3H), 0.04 (s, 3H).

¹³C NMR (125 MHz, DMSO-d₆) δ 180.4, 154.8, 148.5, 148.3, 146.3, 146.0, 137.1, 132.6, 131.6, 131.0, 130.0, 128.3, 127.3, 120.59, 83.9, 83.4, 78.5, 76.8, 62.7, 35.4, 31.7, 28.8, 26.2, 21.7, 21.4, 19.4, 19.2, 18.4, 14.4, −5.1, −5.1.

### Step 3: Ketone Synthesis

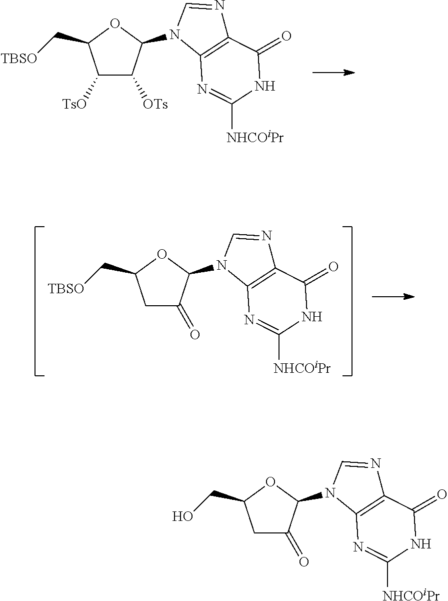

A first reaction vessel was charged with i-PrOH (3.88 L, 50.30 mol) and THF (9.75 L, 1.5 vol) at RT and placed under N₂ before being cooled to −15° C. n-Butyllithium (19.18 L, 47.90 mol, 2.5M in hexanes) was then added slowly, maintaining internal temperature below 25° C. A second vessel was placed under N₂ and charged with the bis-tosylate (6.5 kg, 7.99 mol, 96 wt %) and CPME (26 L, 4 vol.) before being cooled to −5° C. The solution of lithium isopropoxide from the first reaction vessel was then vacuum transferred to the slurry in the second reaction vessel, and the mixture was warmed to 0° C. and aged for about 18 h. The slurry was cooled to −10° C., and AcOH (2.74 L, 47.90 mol) was added slowly, maintaining internal temperature below 5° C. To this mixture was added DI water (32.5 L, 5 vol.), the phases were separated, and the aqueous phase was removed from reactor. The organic layer was cooled to −10° C., and TFA (3.08 L, 40.00 mol) was added slowly, maintaining the internal temperature below 5° C., followed by trioctylamine (6.99 L, 15.98 mol). The mixture was warmed to 0° C. and aged for about 16 h. The slurry was cooled to −10° C., and trioctylamine (10.48 L, 23.97 mol) was added slowly. The resulting homogenous solution was warmed to 25° C. and seeded with 1 wt % of the ketone (26.7 g, 0.0799 mol) and aged for 18 h. The slurry was filtered, and the cake was completely deliquored. The cake was then slurry washed twice with CPME (3.25 L, 0.5 vol.) and then dried under vacuum with N₂ sweep.

¹H NMR (500 MHz, DMSO-d₆) δ 12.12 (s, 1H), 11.47 (s, 1H), 8.11 (s, 1H), 5.99 (s, 1H), 5.05 (t, J=5.6 Hz, 1H), 4.54 (app. dq, J=8.6, 5.4, 4.4 Hz, 1H), 3.71-3.60 (m, 2H), 2.97 (dd, J=18.5, 8.4 Hz, 1H), 2.85-2.75 (m, 2H), 1.14 (app. d, J=6.8 Hz, 6H).

¹³C NMR (125 MHz, DMSO-d₆) δ 209.0, 180.6, 155.2, 149.0, 148.6, 139.4, 120.4, 81.7, 77.1, 63.8, 38.0, 35.3, 19.4, 19.3.

### Step 4: Ketone Fluorination

NFSI (1.964 kg in 5.5 L THF) was charged into a first reaction vessel. The ketone (1.832 kg) was then charged into a separate reaction vessel, followed by THF (5.5 L), H₂O (0.932 L) and L-leucine amide hydrochloride (259 g). The reaction mixture in the second reaction vessel was agitated at 70 rpm at RT. After 40 min., the reaction temperature was 20° C., and 1.5 L NFSI solution (˜20%) from the first reaction vessel was added to the second reaction vessel, followed immediately by 1.371 kg (NH₄)₂HPO₄. The agitation was set 80 rpm. After 20 min., the remainder of the NFSI from the first reaction vessel was charged into the second reaction vessel over 90 min., and the reaction mixture was left for 2 h at 27° C. THF (200 mL) was added to rinse the bottle, and the mixture became homogenous as the temperature increased to 27.9° C. The agitation was then set to 92 rpm. The reaction mixture was then aged for 42 h.

While the temperature was maintained at 27° C., H₂O (10 vol, 18.32 L) was charged into the reaction mixture over 40 min. The reaction mixture was concentrated by distillation, removing THF in batches. Once the distillation was completed, the slurry was allowed to de-supersaturate at 22° C. overnight.

The reaction mixture was set to agitate at 47 rpm. The reaction mixture was filtered under vacuum. The wet cake was then washed with 11 L H₂O, followed by MeCN (2×5.5 L). The wet cake was then dried under N₂ sweep for a period of two and a half days.

¹H NMR (500 MHz, DMSO-d₆:D₂O (5:1)) δ 12.08 (s, 1H), 11.69 (s, 1H), 8.02 (s, 1H), 7.00 (br s, 2H), 5.85 (d, J=1.9 Hz, 1H), 5.15 (br s, 1H), 4.83 (dd, J=53.6, 2.7 Hz, 1H), 4.06 (dddd, J=26.2, 8.3, 2.9, 2.8 Hz, 1H), 3.70-3.63 (m, 2H), 2.77 (sept, J=6.8 Hz, 1H), 1.12 (d, J=6.7 Hz, 6H).

¹³C NMR (126 MHz, DMSO-d₆:D₂O (5:1)) δ 180.7, 155.6, 149.6, 148.3, 139.7, 119.5, 97.2 (d, J=17.8 Hz), 93.4 (d, J=188.7 Hz), 86.1, 81.8 (d, J=23.2 Hz), 60.8 (d, J=7.6 Hz), 35.3, 19.2, 19.1.

¹⁹F NMR (470 MHz, DMSO-d₆:D₂O (5:1)) δ-189.1 (dd, J=53.6, 26.1 Hz).

### Step 5: Chemical Ketone Reduction to 3′-FG

A first reaction vessel was charged with HOAc (2.8 L, 2.0 vol) and MeCN (4.2 L, 3.0 vol) followed by STAB (2.30 kg, 3.0 eq). The walls of the first reaction vessel were rinsed with MeCN (2.8 L, 2.0V). The resulting solution had an internal temperature of 14° C. and was heated to 22° C. over 1 h. The resulting solution was then stirred for 3 h at RT.

A second reaction vessel was charged with HOAc (4.2 L, 3 vol.) and MeCN (6.3 L, 4.5 vol.) followed by the fluorinated ketone (1.40 kg, 3.0 eq.). The vessel walls were rinsed with MeCN (2.1 L, 1.5 vol.). The resulting slurry was heated to 35° C. over 40 min. The solution of STAB from the first reaction vessel was added to the slurry over approximately 2 h. The resulting slurry was stirred for 2 h at 35° C. to 40° C., before the slurry was cooled to 25° C. over 30 min. MeOH (2.8 L, 2 vol.) was added over 1 h, and the resulting solution was allowed to stir for 13.5 h at RT.

The reaction vessel was placed under vacuum for distillation, and the temperature was set to 50° C., with distillation starting when the internal temperature reached to 35° C. Distillation was continued until total ˜4 vol. (5.6 L) of the reaction mixture remained. DI water (2.8 L) was added over 6 min when internal temperature reached 55° C. The walls were washed with water. (NH₄)₂SO₄ (2.8 L, 2 vol.) was added over 20 min to the washed reaction solution.

The reaction mixture was aged for 40 min. Following aging, (NH₄)₂SO₄ (22.4 L, 16 vol.) was added over 4 h, and the slurry was aged again for 2 h at 55° C. The reaction mixture was cooled to RT over 5 h, and then aged at RT for 5.5 h.

After aging, the reaction mixture was filtered, and the filter cake was washed with 4.3 L of H₂O:MeOH (3:1) twice. The cake was then dried under N₂ sweep and vacuum.

¹H NMR (500M Hz, DMSO-d₆) δ 11.68 (s, 2H), 8.27 (s, 1H), 5.96 (d, J=5.4 Hz, 1H), 5.83 (d, J=8.1 Hz, 1H), 5.22 (t, J=5.4 Hz, 1H), 5.07 (dd, J=54.3, 4.1 Hz, 1H), 4.77 (dddd, 27.3, 8.1, 4.1, 4.1 Hz, 1H), 4.25 (dddd, J=27.2, 8.1, 4.1 Hz, 4.1 Hz, 1H), 3.61 (m, 2H), 2.75 (sept, J=6.8 Hz, 1H), 1.12 (d, J=6.8 Hz, 6H).

¹³C NMR (125 MHz, DMSO-d₆) δ 180.2, 154.8, 149.3, 148.4, 137.4, 120.1, 92.8 (d, J=183 Hz), 85.0, 83.6 (d, J=21.1 Hz), 83.5, 72.6 (d, J=16.1 Hz), 60.6 (d, J=11.2 Hz), 34.8, 18.8, 18.8.

¹⁹F NMR (500 MHz, DMSO-d₆) δ-197.5.

### Step 6: Biocatalytic Ketone Reduction to 3′-FG (alternative to Step 5)

10 uL of a ketoreductase enzyme that has the amino acid sequence that is SEQ ID NO: 1, as set forth below, was inoculated into 5 mL of Luria-Bretani Broth (culture media for cells), supplemented with 1% glucose and 50 ug/ml of Kanamycin antibiotic and grown overnight for 20-24 h at 30° C., 250 rpm, in a shaking incubator.

(SEQ ID NO: 1)
MHHHHHPATIVVTGGTKGIGRAIVEKFAKEGFTVLTCARTKGDNF
PENVHFFKADLSKKVEVLAFADFIKQTVNQVDILVNNTGWFLPGE
INNEAEGTLEAMIETNLYSAYYLTRALVGDMITKKEGHIFNICSY
ASIVPYTSGGSYCISKTAQLGMSKVLREELKPHHVRVTSILPGA
VLNDSWAKVELPAELFIAPEDIAQIVWTAHCLPSTTVLEEILIRP
QTGDL

5 mL of the overnight culture was used to subculture 250 mL of Terrific Broth growth media (commercially available from ThermoFisher Scientific as Catalog #A1374301) in a 1 L flask. The subculture was allowed to grow at 30° C. for 3 h at 250 rpm, in a shaking incubator. When the OD measures between 0.4 and 0.6, the IPTG was introduced to an IPTG final concentration of 1 Mm (1 mM). The subculture was allowed to grow overnight, for 18-20 h.

After the growth period, the culture was transferred to a centrifuge bottle and centrifuged for 20 min. at 4000 rpm at 4° C. Following centrifuge, the supernatant was discarded. The cell pellets were resuspended in 50 mM sodium phosphate buffer (pH=7).

The cells from the resuspended cell pellets were lysed using a microfluidizer, and the cell lysate was collected and centrifuged for 60 min. at 10000 rpm at 4° C. The supernatant was transferred to a petri dish and frozen at −80° C. for a minimum of 2 h. Samples were optionally lyophilized using a standard automated protocol.

20 mg of a commercially available ketoreductase enzyme (KRED-P1 B10, commercially available from CODEXIS, Inc.) added to a reaction vessel, along with NADPH (20 mg), a ketoreductase enzyme that can be represented by SEQ ID NO: 1, as set forth above (250 mg, harvested from the subculture), and fluoroketone (250 mg, step 4 above). 10 mL of phosphate buffer (0.1M, pH=6.0) and 1 mL IPA were added to the reaction vessel. The temperature was set at 30° C., and the reaction mixture was stirred at 350 rpm. After 20 h, the mixture was cooled to 15° C. NaCl (2 g) was added to the reaction vessel, and the reaction mixture was allowed to de-supersaturate overnight. The solids were filtered and washed with 2.5 mL (10 vol.) of water. The wet cake was placed into a 50° C. vacuum oven to dry overnight.

### Step 7: Thiophosphorylation

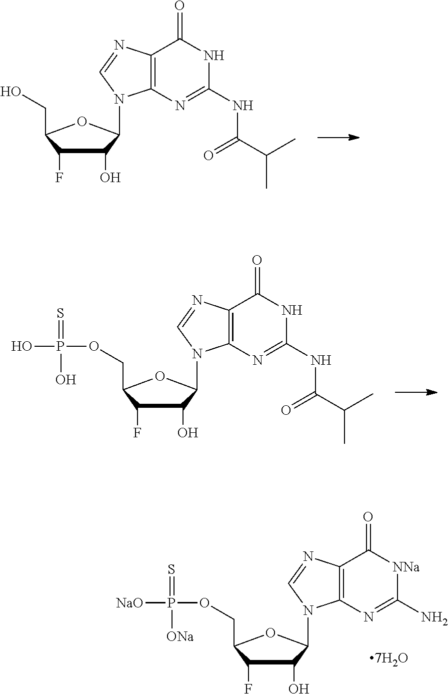

A reaction vessel at 22° C. was charged with THF (16 L). Quinine (234 g, 0.72 mol, 0.25 equiv) was charged into the reaction vessel, followed by 2,6-lutidine (463 g, 4.32 mol, 1.5 equiv), and 3′-FG (1100 g, 2.88 mol, 1.0 equiv, step 5 above). THF (6 L) was used to rinse the sides of the reaction vessel, and the temperature was set to 0° C. PSCl₃ (658 g, 3.89 mol, 1.35 equiv) was charged, maintaining the temperature below 2° C. The reaction mixture was stirred at 80 rpm for approximately 40 h at 0° C. The reaction progress was monitored by UPLC analysis; once 96% conversion had been obtained, the reaction temperature was adjusted to −10° C. H₂O (2.2 L) was added dropwise, maintaining the temperature below 0° C. After the addition, the temperature was adjusted to 25° C., and the reaction mixture was held at this temperature for 1 h.

The THF was removed in vacuo. After THF removal (at least 17 vol.), the vacuum was broken, and the temperature was set to 25° C. MeOH (11 L) was charged into the reaction vessel, and the temperature was adjusted to −10° C. Aqueous NaOH (50 wt %) was diluted with H₂O (1.1 L) and charged into the reaction vessel, maintaining the temperature below 25° C.

The temperature was then adjusted to 45° C., and, after 90 min, the reaction mixture was seeded with 3′-F-thio-GMP (1 wt %, 11 g). The mixture was held at 45° C. for 5 h, then cooled to 20° C. over 5 h, and held at 20° C. THF (1.8 L, 1.6V) added over 45 min at 20° C., and the mixture was agitated for 3 h. The mixture was then filtered, and the wet cake was washed with 10:4:2 MeOH:THF:H₂O (10 L). The cake was then washed with THF (10 L), and the cake was dried under vacuum under a sweep of humidified N₂.

¹H NMR (500 MHz, D₂O): δ 8.17 (s, 1H), 5.94 (d, J=8.4 Hz, 1H), 5.30 (dd, J=54.2, 4.3 Hz, 1H), 4.93 (ddd, J=25.9, 8.4, 4.3 Hz, 1H), 4.63-4.54 (m, 1H), 4.08-4.00 (m, 1H), 3.97-3.89 (m, 1H).

¹³C NMR (126 MHz, D₂O): 167.7, 161.0, 152.15, 135.93, 117.41, 92.53-93.96 (d), 84.68, 82.95 (m), 73.25 (m), 63.50 (m).

¹⁹F NMR (470 MHz, D₂O) 6-197.9.

³¹P NMR (203 MHz, D₂O) δ 43.31.

### Preparatory Example 2: 3′F-thio-GTP from 3′F-thio-GMP Intermediate

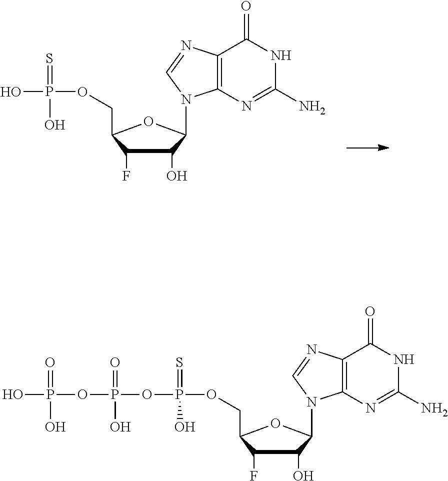

To a 200 mL reactor was charged 3′F-thio-GMP (2.5 g, 73.5 wt %, 1.0 eq), followed by addition of AcP-Li/Li (1.73 g, 94.9 wt %, 2.5 eq). Next, 2′F-thio-ATP (140 mg, 81 wt %, 0.05 eq) was charged at 25° C., followed by addition of water (50 mL) and EtOH (12.5 mL), respectively. To the resulting mixture was added MgCl₂ (4.33 mL, 1M, 1.0 eq) and KCl (2.16 mL, 3M, 1.5 eq). Then, pH was adjusted to 6.4-6.6 using HCl (9M) solution while the solution was agitated 25° C. Next, a kinase enzyme that has the amino acid sequence that is SEQ ID NO: 2 (26.5 mg), as set forth below, and a kinase enzyme that has the amino acid sequence that is SEQ ID NO: 3 (26.5 mg), as set forth below, were charged to the reactor. The mixture was stirred at 25° C. until completion (6-8 h) using overhead stirrer. After completion (˜97% conversion), HOAc was added (1.8 mL) to adjust the pH to 4.3-5.0, followed by slow addition of MeCN (˜250 mL) to precipitate the product. At the end, the slurry was filtered, and the wet cake was washed with the same solvent ratio as mother liquor (ML) to afford the product, which was dried under vacuum to give 3′F-thio-GTP.

(SEQ ID NO: 2)
MALPRPVVICGPSGSGKSTLYNKLLKEFPGVFQLSVSHTTRQPRP
GELNGREYHFINRDQFQENIKQGDFLEWAEFSGNIYGTSKKALEE
VQSNNVIPILDIDTQGVRNVKKASLEAVYIFIKPPSIDVLEKRLR
SRKTETEEALQKRLSAARNELEYGLKPGNFQHIITNDDLDVAYEK
LKGILIKSQMPLAMATGSSSSVVNSFLDKPAASATTVNSSSQD.
(SEQ ID NO: 3)
MHHHHHHSSSESIRMVLIGPPGAGKGTQAPNLQERFHACHLATGD
MLRSQIAKGTQLGLEAKKIMDQGGLVSDDIMVNMIKDELTNNPAC
KNGFILDGFPRTIPQAEKLDQMLKEQGTPLEKAVELKIDDELLPA
RITGRLIHPASGRSYHKIFNPPKEDMKDDVTGEALVQRSDDNADA
LKKRLAAYHKQTEPVVDFYKKTGIWAGVDASQPPATVWADILNKL
GKD

The product was redissolved in water (40 mL), and the pH was adjusted with KOH to pH 7.0. To the solution, KCl (800 μl, 3M, 0.1 eq) was added, followed by HOAc (1.6 mL). MeCN (57 mL) was added slowly to precipitate 3′F-thio-GTP. The slurry was filtered, and the wet cake was washed with the same solvent ratio as mother liquor (ML) and dried under vacuum to give 3′F-thio-GTP.

### Preparatory Example 3: Synthesis of (O-{[(2R,3R,4S,5R)-5-(6-amino-9H-purin-9-yl)-4-fluoro-3-hydroxyoxolan-2-yl]methyl}O,O-dihydrogen phosphorothioate

### Step 1: Synthesis of Trimethyl(((2R,3S)-3-((trimethylsilyl)oxy)-2,3-dihydrofuran-2-yl)methoxy) silane from Thymidine

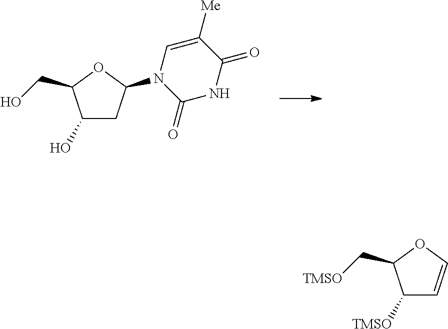

A 100 L vessel was charged with toluene (14.5 L), thymidine (4825 g, 20 mol), 2,6-lutidine (1081 g, 0.400 mol), PTPI (90 g, 0.200 mol) and heptane (33.8 L). The mixture was heated to 90° C. To this, BSA (17.4 kg, 85.6 mol) was added over 30 min. The mixture was heated to 100° C. and stirred at 100-107° C. for 3 h. After cooling to room temperature, the reaction mixture was transferred to another TOOL reactor containing i-PrOH (12.3 L, 161 mol) slowly (204 ml/min). Toluene (1 L) was used to rinse and transfer any remaining material in the first reactor. The resulting slurry was stirred at 35° C. for 2 h, then cooled to 10° C. and aged at that temperature overnight. The resulting slurry was filtered, and the filter cake was washed with heptane (20.0 L). The combined filtrates were passed through a plug of basic alumina and transferred to a 100 L vessel. The resulting solution was concentrated under vacuum to the total volume of 24 L, which was used in the subsequent reaction without further purification.

¹H NMR (400 MHz, CD₂Cl₂); δ 6.50 (dd, J=2.7, 1.1 Hz, 1H), 5.06 (t, J=2.7 Hz, 1H), 4.84 (td, J=2.7, 1.0 Hz, 1H), 4.28 (ddd, J=6.7, 6.1, 2.7 Hz, 1H), 3.67 (dd, J=10.6, 6.1 Hz, 1H), 3.47 (dd, J=10.6, 6.7 Hz, 1H), 0.17 (s, 9H), 0.16 (s, 9H).

### Step 1, alternate route: Synthesis of Trimethyl(((2R,3S)-3-((trimethylsilyl)oxy)-2,3-dihydrofuran-2-yl)methoxy) silane from 2′-Deoxyuridine

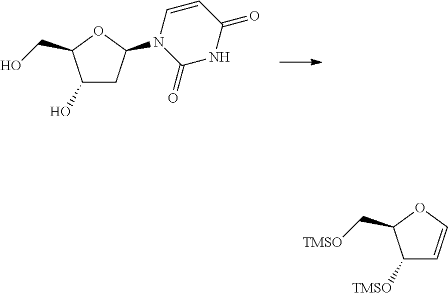

In an 8 mL vial, dry 2′-deoxyuridine (1 mmol), PTPI (0.01 eq, 5 mg), 2,6-lutidine (0.5 eq, 58 μL), 1 mL heptane, 1 mL toluene, and 3.5 eq. of BSA was added under nitrogen atmosphere. The reaction was stirred at 100° C. for 3 h. Reaction progress was monitored via HPLC by the presence of starting material.

### Step 1, alternate route: Synthesis of 2-tert-butyl(((2R,3S)-3-((tert-butyldimethylsilyl)oxy)-2,3-dihydrofuran-2-yl)methoxy)dimethylsilane (2-TBS)

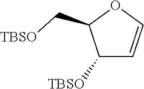 [SMILES: CC(C)(C)[Si](C)(C)OC[C@H]1OC=C[C@@H]1O[Si](C)(C)C(C)(C)C]

In a 2 L flask was charged ammonium sulfate (5.38 g, 40.7 mmol), bis(tert-butyldimethylsilyl)thymidine (100 g, 203 mmol), and 2,6-di-tert-butyl-4-methylphenol (0.045 g, 0.203 mmol). HMDS (141 mL, 671 mmol) and heptane (1000 mL) were subsequently added, and the reaction mixture was heated to reflux (140° C. external bath) under nitrogen atmosphere. After 34 h, the reaction mixture was cooled to ambient temperature. 2,4,6-trimethylpyridine (13.55 mL, 102 mmol) was added followed by ethanol (35.6 mL, 610 mmol) via syringe pump over 2 h. The resulting slurry was then filtered, and the cake was washed with CPME (4×150 mL). The filtrate was concentrated to provide 2-TBS (57.14 g, 166 mmol) by quantitative NMR analysis.

¹H NMR (500 MHz, CDCl₃) δ 6.47 (dd, J=2.6, 0.8 Hz, 1H), 5.01 (t, J=2.6 Hz, 1H), 4.87 (td, J=2.6, 0.8 Hz, 1H), 4.29 (td, J=6.0, 2.8 Hz, 1H), 3.69 (dd, J=10.7, 5.7 Hz, 1H), 3.51 (dd, J=10.7, 6.3 Hz, 1H), 0.90 (s, 9H), 0.89 (s, 9H), 0.09 (s, 3H), 0.09 (s, 3H), 0.07 (s, 3H), 0.06 (s, 3H).

¹³C NMR (126 MHz, CDCl₃) δ 149.1, 103.6, 89.1, 76.1, 63.0, 26.1, 26.0, 18.5, 18.2, −4.1, −4.3, −5.2, −5.2.

### Step 2: Synthesis of N-((2S,3S,4R,5R)-4-((tert-butyldimethylsilyl)oxy)-5-(((tert-butyldimethylsilyl)oxy)methyl)-3-fluorotetrahydrofuran-2-yl)-N-(phenylsulfonyl) benzenesulfonamide (3-TBS)

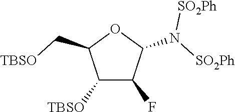

In a 1 L flask was charged the crude 2-TBS (1.0 equiv, 82.95 g, 166 mmol) and CPME (263 mL). Additionally, 2,4,6-trimethylpyridine (4.53 mL, 34.2 mmol), BSTFA (22.02 mL, 83 mmol), and NFSI (68.0 g, 216 mmol) were added to the reaction mixture. The resulting mixture was warmed to 65° C. and stirred for 20 h. After cooling to ambient temperature, heptane (286 mL) was added, and the reaction mixture was stirred for 1.75 h at ambient temperature. The resulting slurry was filtered, and the cake was washed with CPME:heptane (1:1, 286 mL). The filtrate was subsequently concentrated under vacuum. Heptane (286 mL) was added to the concentrated crude material, and the mixture was heated to 70° C. The mixture was filtered while hot into a 1 L flask, and the filtrate was crystallized while being slowly cooled to ambient temperature. The resulting slurry was further cooled to −30 to −35° C. and filtered. After drying under vacuum, the desired DBSI adduct 3-TBS (94.63 g, 138 mmol) was collected.

¹H NMR (500 MHz, CDCl₃) δ 8.04 (dd, J=8.5, 1.1 Hz, 4H), 7.64 (t, J=7.5 Hz, 2H), 7.54 (t, J=7.9 Hz, 4H), 6.00 (dd, J=16.5, 5.9 Hz, 1H), 5.67 (ddd, J=57.2, 6.3, 6.3 Hz, 1H), 4.48 (ddd, J=21.3, 8.9, 6.7 Hz, 1H), 4.39-4.24 (m, 1H), 3.78 (ddd, J=12.0, 1.8, 1.8 Hz, 1H), 3.65 (dd, J=12.0, 3.1 Hz, 1H), 0.92 (s, 9H), 0.88 (s, 9H), 0.10 (s, 3H), 0.08 (s, 3H), 0.05 (s, 3H), 0.05 (s, 3H).

¹³C NMR (126 MHz, CDCl₃) δ 140.6, 134.0, 129.1, 128.4, 99.7 (d, J=188.1 Hz), 92.2 (d, J=36.8 Hz), 83.4 (d, J=9.9 Hz), 73.0 (d, J=20.9 Hz), 61.3, 26.0, 25.7, 18.5, 18.0, −4.5, −5.0, −5.1, −5.3.

¹⁹F NMR (500 MHz, CDCl₃) δ-195.0.

### Step 3: Synthesis of 1-((2R,4S,5R)-4-((tert-butyldimethylsilyl)oxy)-5-(((tert-butyldimethylsilyl) oxy) methyl)tetrahydrofuran-2-yl)-5-methylpyrimidine-2,4(1H,3H)-dione

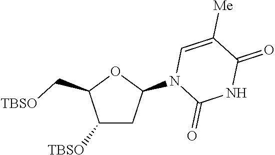 [SMILES: Cc1cn([C@H]2C[C@H](O[Si](C)(C)C(C)(C)C)[C@@H](CO[Si](C)(C)C(C)(C)C)O2)c(=O)[nH]c1=O]

While under a nitrogen atmosphere, thymidine (12.1 g, 50 mmol), imidazole (2.5 equiv, 8.5 g, 125 mmol), tert-butyldimethylsilyl chloride (2.2 equiv, 16.6 g, 110 mmol), DMF (20 mL), and DMAP (0.01 equiv, 0.061 g, 0.5 mmol) were added to a 200 mL round-bottom flask, and the resulting mixture was stirred for 1 h at ambient temperature. The reaction was determined to be complete by HPLC. Subsequent addition of 100 mL water was followed by stirring at ambient temperature for 1 h. Filtration of the slurry was performed, and the cake was washed with 200 mL water. The cake was dissolved in 100 mL MTBE, and the solution was washed with 100 mL water and dried over magnesium sulfate. The filtered MTBE solution was evaporated to approximately 30 mL, diluted with 30 mL hexanes and 80 mL heptane and evaporated to approximately 100 mL. The residue was cooled to 0° C. over 2 h, and crystallization was observed to occur. The slurry was filtered and washed with 30 mL 9:1 hexanes:MTBE and subsequently with 50 mL hexanes. The solid was dried under a nitrogen stream to provide 1-((2R,4S,5R)-4-((tert-butyldimethylsilyl) oxy)-5-(((tert-butyldimethylsilyl)oxy)methyl) tetrahydrofuran-2-yl)-5-methylpyrimidine-2,4(1H,3H)-dione (21 g, 44.6 mmol).

¹H NMR (500 MHz, CDCl₃) δ 8.35 (s, 1H), 7.47 (d, J=1.2 Hz, 1H), 6.33 (dd, J=7.9, 5.8 Hz, 1H), 4.40 (ddd, J=5.6, 2.5, 2.5 Hz, 1H), 3.93 (ddd, J=2.5, 2.5, 2.5 Hz, 1H), 3.87 (dd, J=11.4, 2.6 Hz, 1H), 3.76 (dd, J=11.4, 2.4 Hz, 1H), 2.25 (ddd, J=13.1, 5.8, 2.6 Hz, 1H), 2.00 (ddd, J=13.3, 7.9, 6.1 Hz, 1H), 1.92 (d, J=1.1 Hz, 3H), 0.93 (s, 9H), 0.89 (s, 9H), 0.11 (s, 3H), 0.11 (s, 3H), 0.08 (s, 3H), 0.07 (s, 3H),

¹³C NMR (126 MHz, CDCl₃) δ 164.3, 150.7, 135.8, 111.2, 88.2, 85.2, 72.6, 63.4, 41.8, 26.3, 26.1, 18.8, 18.4, 12.9, −4.3, −4.5, −5.0, −5.1.

### Step 4: Synthesis of Piv-protected (2R,3R,4R,5R)-5-(6-amino-9H-purin-9-yl)-4-fluoro-2-(hydroxymethyl)tetrahydrofuran-3-ol

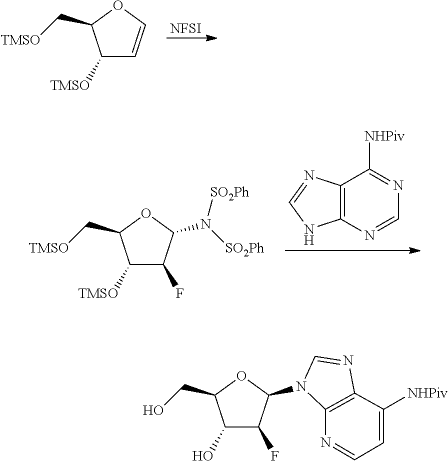

To a 100 L reactor was charged the crude toluene stream for trimethyl(((2R,3S)-3-((trimethylsilyl)oxy)-2,3-dihydrofuran-2-yl)methoxy)silane (10.2 kg, 13.4 mol), which contained 0.36 equivalents lutidine and 2 vol toluene. To this was added toluene (8.75 L), 2,6-lutidine (0.563 L, 4.84 mol), and BSTFA (0.178 L, 0.672 mol), and the resulting mixture was warmed to 65° C. N-fluorobenzenesulfonimide (NFSI) (4.66 kg, 14.77 mol) was added portionwise, then toluene (1.75 L) was used to rinse the sides of the reactor. The reaction mixture was stirred at 65° C. until trimethyl(((2R,3S)-3-((trimethylsilyl)oxy)-2,3-dihydrofuran-2-yl)methoxy)silane was consumed judged by NMR analysis, after which 2,6-lutidine (0.782 L, 6.72 mol), ethyl acetate (50.75 L) and N-(9H-purin-6-yl)pivalamide (2.88 kg, 12.76 mol) were added. An additional 1.75 L of ethyl acetate was used to rinse the sides of the reactor. The resulting mixture was warmed to 75° C. and stirred for overnight. The crude reaction mixture was then concentrated under vacuum to a total volume of 35 L. The resulting slurry was filtered, and the filter cake was washed with ethyl acetate (17.5 L, 5 vol). The filtrate was transferred to a 50 L reactor while continuously evaporating under vacuum to a total volume of 17.5 L. To this, ethanol (5.25 L), 2,6-lutidine (0.313 L, 2.69 mol), and TFA (103 ml, 1.34 mol) were added to start desilylation. Vacuum distillation while feeding 21 L of 3.8:1 v/v EtOAc:EtOH was conducted to aid the desilylation.

When the desilylation was achieved with >90% conversion 17.5 L EtOAc was fed into the reactor while distilling away excess EtOH. An additional continuous vacuum distillation was performed with 3.5 L toluene feed while the mixture was concentrated to the total volume of 17.5 L. After the distillation was completed, the reaction mixture was stirred at room temperature overnight to crystallize. The product was collected by filtration rinsing with 21 L of 2:10:88 v/v/v EtOH:tol:EtOAc. Total mass was 3.16 kg.

¹H NMR (500 MHz, DMSO-d₆) δ 10.19 (s, 1H), 8.72 (s, 1H), 8.56 (d, J=1.9 Hz, 1H), 7.30-7.10 (toluene, m, 5H), 6.55 (dd, J=13.4, 4.7 Hz, 1H), 5.99 (bs, 1H), 5.29 (ddd, J=52.6, 4.3, 4.3 Hz, 1H), 4.49 (ddd, J=19.1, 4.6, 4.6, 1H), 3.89 (ddd, J=4.8, 4.8, 4.8 Hz, 1H), 3.72 (dd, J=12.1, 3.1 Hz, 1H), 3.66 (dd, J=12.0, 5.1 Hz, 1H), 2.30 (toluene, s, 3H), 1.28 (s, 9H).

¹³C NMR (124 MHz, DMSO-d₆) δ 176.3, 151.8, 151.7, 150.4, 142.8 (d, J=3.8 Hz), 137.4 (toluene), 128.9 (toluene), 128.2 (toluene), 125.2 (toluene), 125.1, 95.4 (d, J=192.4 Hz), 83.5 (d, J=5.7 Hz), 81.5 (d, J=17.0 Hz), 72.5 (d, J=23.0 Hz), 60.3, 26.9, 21.0 (toluene).

¹⁹F NMR (470 MHz, DMSO-d₆) δ 197.9.

### Step 5: Synthesis of (O-{[(2R,3R,4S,5R)-5-(6-amino-9H-purin-9-yl)-4-fluoro-3-hydroxyoxolan-2-yl]methyl}O,O-dihydrogen phosphorothioate from (2R,3R,4R,5R)-5-(6-amino-9H-purin-9-yl)-4-fluoro-2-(hydroxymethyl)tetrahydrofuran-3-ol

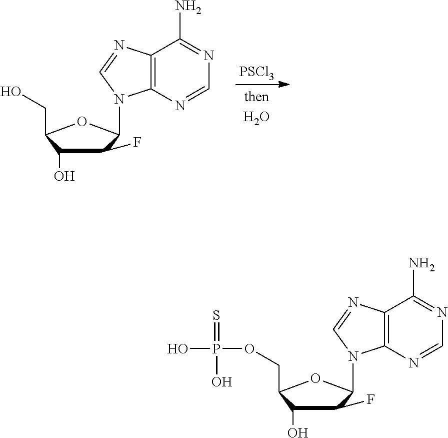

(2R,3R,4R,5R)-5-(6-amino-9H-purin-9-yl)-4-fluoro-2-(hydroxymethyl) tetrahydrofuran-3-ol (50 g, 186 mmol) was azeotroped with 3×100 mL of dry pyridine and was dissolved in 500 mL (10 vol) of dry pyridine (KF=128 ppm). The pyridine solution was cooled to 0° C. for 1 h. Thiophosphoryl chloride (1.04 eq) was added dropwise at 0° C. over 10 min. The reaction was stirred at 0° C. for 80 min, with constant monitoring by UPLC. The reaction was filtered to remove the excess starting material. Water (10 eq) was then added to the filtrate at 0° C. and was slowly warmed to room temperature. The reaction was allowed to stir for an additional 30 min at room temperature. The volatiles were removed in vacuo, and the product was dissolved in 500 mL of water. The solution pH was 4. The solution was filtered, and the filtrate was stirred while 12M HCl was added until the pH of the solution was 0 (about 35 mL). The resulting slurry was allowed to stir at room temperature overnight (˜16 h). Then the slurry was allowed to settle for 1 h. The slurry was then filtered, and the filter cake was washed with 200 mL of water. The washed cake was allowed to dry over a stream of nitrogen overnight (29.9 g).

¹H NMR (500 MHz, DMSO-d₆) (=8.26 (d, J=1.9 Hz, 1H), 8.21 (s, 1H), 7.55 (br s, 2H), 6.46 (dd, J=15.0, 4.5 Hz, 1H), 5.24 (dt, J=52.4, 4.1 Hz, 1H), 4.51 (dt, J=18.1, 4.2 Hz, 1H), 4.22-4.04 (m, 3H).

¹³C NMR (125 MHz, DMSO-d₆): (=155.9, 152.6, 149.5, 140.3 (d, J=4.1 Hz), 118.7, 95.4 (d, J=191.9 Hz), 82.1 (d, J=16.7 Hz), 81.8 (dd, J=9.3, 5.3 Hz), 73.5 (d, J=23.7 Hz), 65.50 (dd, J=4.6, 1.8 Hz).

¹⁹F NMR (470 MHz, DMSO-d₆): (=−197.80 (ddd, J=52.7, 16.6, 16.6 Hz, 1F).

³¹P NMR (202 MHz, DMSO-d₆): 3=59.49 (dd, J=7.4, 7.4 Hz, 1P).

### Step 5, alternate route: Synthesis of (O-{[(2R,3R,4S,5R)-5-(6-amino-9H-purin-9-yl)-4-fluoro-3-hydroxyoxolan-2-yl]methyl}O,O-dihydrogen phosphorothioate from Piv-protected (2R,3R,4R,5R)-5-(6-amino-9H-purin-9-yl)-4-fluoro-2-(hydroxymethyl)tetrahydrofuran-3-ol

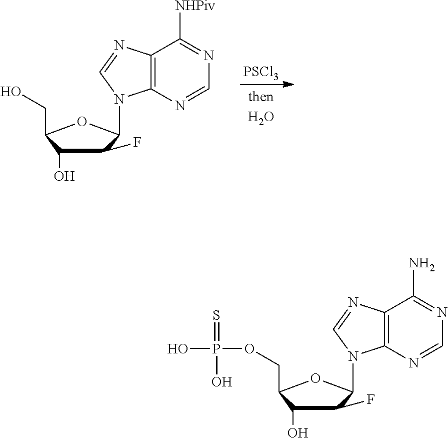

In a 50 L reactor was charged triethylphosphate (4 vol, 8.58 L), Piv-protected (2R,3R,4R,5R)-5-(6-amino-9H-purin-9-yl)-4-fluoro-2-(hydroxymethyl)tetrahydrofuran-3-ol (2.5 kg, 85.75 wt %) followed by the remaining triethylphosphate (1 vol, 2.14 L) washing the sides of the vessel. To this, 2,6-lutidine (3 eq, 1.97 kg) and pyridine (0.3 eq, 144 g) were charged, and the resulting mixture was cooled to −20° C. Then thiophosphoryl chloride (1.835 kg, 1.75 eq.) was added slowly over 1 h. The reaction mixture was aged at −20° C. for overnight, after which additional thiophosphoryl chloride (32 mL, 0.05 eq.) was added. Water (8 eq, 0.87 L) was added dropwise over 1 h to quench the reaction. Additional water (32 eq, 3.5 L) was added dropwise over 1 h, then the resulting mixture was warmed to 50° C. and aged at that temperature for 3 h. After pivaloyl group was removed judged by HPLC analysis, the mixture was cooled to 30° C., and added water (9 vol, 19.3 L) to crystallize the product while cooling to 0° C. slowly. The product was collected by filtration rinsing with water (12.5 L) then dried under vacuum with nitrogen sweep. The resulting product (1.971 kg, 88.65 wt %) was then collected and stored under ambient temperature.

¹H NMR (500 MHz, DMSO-d₆) δ=8.26 (d, J=1.9 Hz, 1H), 8.21 (s, 1H), 7.55 (br s, 2H), 6.46 (dd, J=15.0, 4.5 Hz, 1H), 5.24 (dt, J=52.4, 4.1 Hz, 1H), 4.51 (dt, J=18.1, 4.2 Hz, 1H), 4.22-4.04 (m, 3H).

¹³C NMR (125 MHz, DMSO-d₆): δ=155.9, 152.6, 149.5, 140.3 (d, J=4.1 Hz), 118.7, 95.4 (d, J=191.9 Hz), 82.1 (d, J=16.7 Hz), 81.8 (dd, J=9.3, 5.3 Hz), 73.5 (d, J=23.7 Hz), 65.50 (dd, J=4.6, 1.8 Hz).

¹⁹F NMR (470 MHz, DMSO-d₆): δ=−197.80 (ddd, J=52.7, 16.6, 16.6 Hz, 1F).

³¹P NMR (202 MHz, DMSO-d₆): δ=59.49 (dd, J=7.4, 7.4 Hz, 1P).

### Preparatory Example 4: Synthesis of 2′F-Thio-ATP from (O-{[(2R,3R,4S,5R)-5-(6-amino-9H-purin-9-yl)-4-fluoro-3-hydroxyoxolan-2-yl]methyl}O,O-dihydrogen phosphorothioate

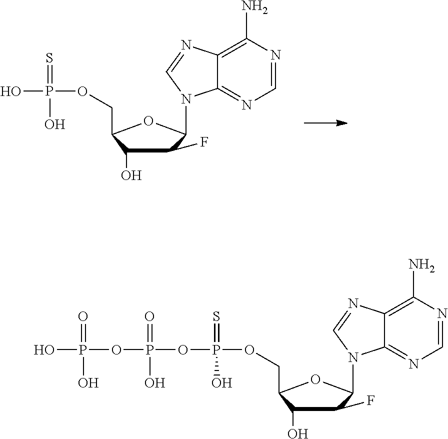

To a 1 L reactor was charged 2′F-thio-AMP (23.3 g, 86 wt %), 2′F-thio-ATP disodium salt, tetrahydrate (1 g, 85 wt %) and 0.9M aq. solution of acetyl phosphate, disodium (3.6 eq, 219 mL). The reaction solution was added 1M aq solution of MgCl₂·(H₂O)₆ solution (0.125 eq, 6.9 mL), and the pH of the reaction mixture was adjusted to 6.5 with addition of NaOH. The reaction volume was diluted to 500 mL with water. An adenylate kinase enzyme that has the amino acid sequence that is SEQ ID NO: 4 as set forth below (100 mg) and a kinase enzyme that has the amino acid sequence that is SEQ ID NO: 5 as set forth below (200 mg) were charged to the reaction vessel, and the reaction mixture was stirred at 500 rpm at ambient temperature. After 6 h, the reaction was quenched with 37% aq. solution of HCl (40 mL) to bring the pH to 2. The resulting slurry was filtered, and the filtrate was transferred into 3 L vessel with an overhead stirrer rate of 270 rpm. The filtered solution was charged sodium chloride (2.0 eq, 6.41 g). EtOH (505 mL) was charged to the reaction mixture, and 2′F-Thio-ATP, disodium salt, tetrahydrate was added as seeds. Once seed bed is formed, the crystal slurry was stirred overnight at 270 rpm. After overnight aging, the slurry was charged another portion of EtOH (130 mL) over 2 h via addition funnel. The reaction vessel was cooled to 4° C. Another portion of EtOH (500 mL) was charged over 4 h via addition funnel to reach EtOH/water ratio of approximately 2:1. The slurry was filtered, and the wet cake was washed with cold 2:1 EtOH/water solution (4×20 mL), cold EtOH (3×20 mL). The resulting wet cake was dried under vacuum with positive nitrogen pressure overnight to yield 2′F-Thio-ATP (31.3 g).

(SEQ ID NO: 4)
MHHHHHHSSSESIRMVLIGPPGAGKGTQSPNLQERFHACHLGTGD
MLRSQHAKGTQLGLEAKKIMDQGGLVSDDIMVNMIKDELTNNPAC
KNGFILDGFPRTIPQAMKLEQMLKEQGTPLEKAVELKIDDELLPA
RITGRLIHPASGRSYHKIFNPPKEDMKDDVTGEALVQRSDDNADA
LKKRLAAYHKQTEPVVDFYKKAGIWAGVDASQPVATVWADILNKL
GKD
(SEQ ID NO: 5)
MGSHHHHHGSRVLVINSGSSSIKYQLIEMEGEKVLCKGIAERIGI
EGSRLVHRVGDEKHVIERELPDHEEALKLILNTLVDEKLGVIKDL
KEIDAVGHRVVHGGERFKESVLVDEEVLKAIEEVSPLAPLHNPAN
LMGIKVAMKLLPGVPNVAVFDTAFHQTIPQKAYLYAIPYEYYEK
YKIRRYGFHGTSHRYVSKRAAEILGKKLEELKIITCHIGNGASVA
AVKYGKCVDTSMGFTPTEGLVMGTRSGDLDPAIPFFIMEKEGIS
PQEMYDILNKKSGVYGLSKGFSSDLRDIEEAALKGDEWCKLVLEI
YDYRIAKYIGAYAAAMNGVDAIVFTAGVCENSPITREDVCSYLEF
LGVKLDKQKNEETIDGKEGIISTPDSRVKVLVVPTNEELMIARDT
KEIVEKIGR

1H NMR (500 MHz, Deuterium Oxide) δ 8.67 (d, J=1.8 Hz, 1H), 8.49 (s, 1H), 6.60 (dd, J=11.7, 4.8 Hz, 1H), 5.40 (dt, J=51.7, 4.6 Hz, 1H), 4.81 (m, 1H), 4.45-4.38 (m, 2H), 4.35 (m, 1H).

¹³C NMR (126 MHz, Deuterium Oxide) δ 151.0 147.9, 146.8, 142.7 (d, J=3.7 Hz), 117.9, 94.3 (d, J=193.8 Hz), 82.5 (d, J=17.1 Hz), 81.7 (dd, J=9.4, 5.8 Hz), 72.2 (d, J=24.6 Hz), 64.3 (d, J=6.3 Hz).

³¹P NMR (203 MHz, Deuterium Oxide) δ 43.92 (d, J=27.0 Hz), −10.88 (d, J=19.4 Hz), −23.94.

### Preparatory Example 5: Preparation of Cobalt-Treated cGAS

500 mL of cGAS whole cell lysate was spun at 5000 G-force at 4° C. for 20 min. The supernatant was discarded, and the insoluble fraction was suspended with 500 mL (1 vol) of ultrapure, deionized, biology-grade water. The resulting mixture was spun at 5000 G-force at 4° C. for 20 min. The resulting supernatant was discarded, and the insoluble fraction was suspended with 500 mL of 0.1M CoSO₄ (1 vol, pH 4-8). The mixture was incubated for 1 h at RT. The resulting mixture was spun at 5000 G-force at 4° C. for 20 min. The resulting supernatant was discarded, and the insoluble fraction was suspended with 500 mL of ultrapure, deionized, biology-grade water (1 vol). The resulting mixture was spun at 5000 G-force at 4° C. for 20 min. The resulting supernatant was discarded, and the insoluble fraction is Co-treated cGAS, which was stored at 4° C. and used directly for the cGAS reaction.

### Preparatory Example 6: Tandem Synthesis of 2′F-Thio-ATP and 3′F-Thio-GTP from Monophosphate Precursors Using Co-Immobilized Enzymes

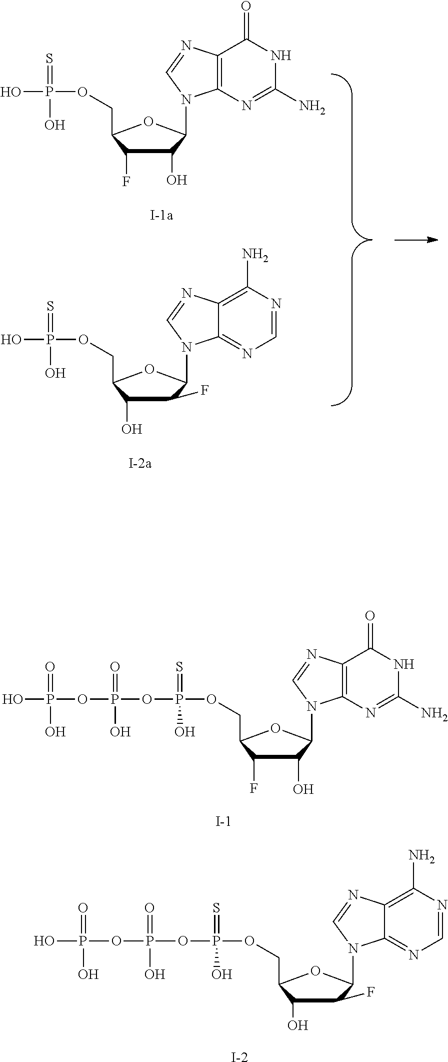

### Step 1: Immobilization

Ni-functionalized chelating resin suspension (commercially available as Bio-rad Nuvia IMAC Ni, 1.8 L, 53 vol % resin solids in 20%/80% EtOH/water) was added to a filter and washed (10 L) with binding buffer (50 mM sodium phosphate buffer; 500 mM NaCl, pH 8) to remove the resin storage solution. The resin was isolated as a cake by filtration, and then re-suspended in the binding buffer (0.75 L) and transferred by funnel into a first reactor (10 L). An addition 0.25 L of binding buffer was used to rinse the transfer vessel, and this liquid was also transferred into the first reactor.

In a second vessel, lyophilized crude cell-free extracts were charged at a pre-determined ratio: a kinase enzyme that has the amino acid sequence that is SEQ ID NO: 6 (21.20 g), as set forth below, a kinase enzyme that has the amino acid sequence that is SEQ ID NO: 4 (16.90 g), as set forth above, and a kinase enzyme that has the amino acid sequence that is SEQ ID NO: 5 (12.70 g), as set forth above, and the extracts were dissolved in binding buffer (1.0 L). Following complete dissolution, the contents of the second vessel were charged into the first reactor and aged overnight at 4° C. with overhead agitation. The resulting mixture was filtered over vacuum yielding a wet cake of immobilized-enzyme on resin. The resulting cake was subsequently washed with 10 L of a modified binding buffer containing imidazole (50 mM sodium phosphate buffer; 500 mM NaCl; 15 mM imidazole, pH 8) and then washed with water (10 L). The washed resin was isolated as a wet cake by filtration, re-suspended in water (1.0 L), and stored at 4° C. prior to use.

(SEQ ID NO: 6)
MHHHHHHALPTPVVICGPSGSGKTTLYNKLLKEFPGVFQLTASHT
TRQPRPGEENGREFHFINRDQFQENIKQGDFLEWAEHSGNLYGTS
KKALEEVQANNVIPILDIDTQGVRTVKKASLEAVYIFIKPPSIEV
LEERLRSRGTETEEALQKRLSAAPNELEYGLKPGNFQHIITNDDL
DVAYEKLKGILIDSQMPLAGATGSSSSVVNSFLDKPAASATTVNS
SSQD

### Step 2: Reaction

In a third reactor (100 L), the following material was charged and held at 25° C.: 25 L water, followed by 3′F-thio-GMP (600 g, 1.0 eq), followed by 1.0 L water to rinse the vessel walls. The mixture was dissolved with overhead agitation and subsequently cooled to 10° C. To the third reactor, 2′F-thio-AMP (283 g, 0.87 eq), 2′F-thio-ATP (5.29 g, 0.01 eq), dilithium acetylphosphate (609 g, 88 wt % purity, 4.5 eq), MgCl₂·5H₂O (374 g, 2.0 eq), and KCl (68.6 g, 1.0 eq) were charged, followed by water (1.0 L) to rinse the walls of the vessel. The resulting mixture was held at 10° C. and briefly agitated. The pH of the solution was then adjusted to approximately 7.3-7.4 using conc. KOH and HCl (5.0 N). Water was added to adjust the final fill volume to 28.15 L.

While continuing to agitate the third reactor, 15% of the immobilized enzyme prepared in Step 1 was aliquoted into a bottle and stored at 4° C., while the remaining 85% of the immobilized enzyme was added to the 50 L reactor, including 500 mL water used to rinse the vessel in which the immobilized enzyme was stored. An additional 500 mL water was added to the reactor to rinse the vessel walls. The mixture was aged for 22 h at 10° C. After the reaction was judged complete by HPLC analysis, the vessel contents were emptied into a filter, and the reaction filtrate was isolated under gentle vacuum and stored at 4° C. or −20° C. for subsequent use.

### Preparatory Example 7: Tandem Synthesis of 2′F-thio-ATP and 3′F-thio-GTP from Monophosphate Precursors Using Independently Immobilized Enzymes

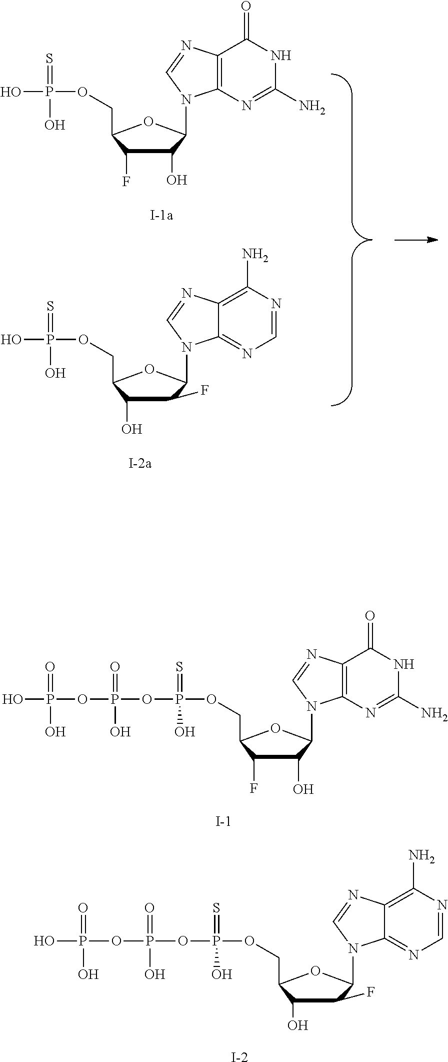

### Step 1: Immobilization

Ni-NTA resin (commercially available as Bio-rad Nuvia IMAC Ni, 2.14 mL of 70 vol % resin slurry) was transferred to a filter, and the storage solution removed by vacuum filtration. Subsequently, the resin was displacement washed with a total of 15 mL binding buffer (50 mM sodium phosphate buffer; 500 mM NaCl, pH 8), resuspended in 3.0 mL binding buffer and transferred to a centrifuge tube, yielding a 50 vol % suspension of resin in binding buffer.

Lyophilized CFE powders of a kinase enzyme that has the amino acid sequence that is SEQ ID NO: 6 (as set forth above), a kinase enzyme that has the amino acid sequence that is SEQ ID NO: 4 (as set forth above), and a kinase enzyme that has the amino acid sequence that is SEQ ID NO: 5 (as set forth above) were separately immobilized as follows: 25 mg of the respective lyophilized CFE was weighed into a vial and resuspended in 0.5 mL binding buffer. To each 1.0 mL of the 50 v % suspension of Ni-NTA resin prepared above was added, followed by an additional 1.0 mL binding buffer. Each vial was closed and mixed at RT for 1 h to complete the immobilization.

Subsequently, the immobilized enzyme-resin from each vial was isolated as follows: the supernatant was decanted, and the resin was washed with a total of 5.0 mL of a modified binding buffer (50 mM sodium phosphate buffer; 500 mM NaCl, 15 mM imidazole, pH 8) followed by 5.0 mL of 1×PBS, the supernatant was decanted, and the resin was resuspended in 1.5 mL water to obtain a 33 vol % slurry of immobilized enzyme resin in water.

### Step 2: Reaction

A reaction master mix was created by charging the following to a vessel: 2′F-Thio-ATP (9.45 mg, 0.05 eq), 2′F-Thio-AMP (111 mg, 0.87 eq), 3′F-Thio-GMP (200 mg, 1.0 eq), dilithium acetyl phosphate (207 mg, 4.25 eq), water (8.0 mL), 1M MgCl₂·6H₂O (604 μL, 2 eq). The pH was adjusted to 7.47 by addition of 2N KOH (145 μL, 0.98 eq) and brought up to 10.0 mL with water. The stock solution was stored at 4° C. until ready for use.

Reactions were performed in a 96-well deep well microplate. To each well, 500p of the reaction master mix was added. The reaction stoichiometry for each experiment was varied by changing the volume of each immobilized enzyme resin charged into the wells, between 0.1 μL and 5.0 μL of each resin.

The plate was sealed and mixed on a thermomixer at 10° C. The reaction progress was assessed at both 16 h and 24 h time points. For each, the reaction mixture was sampled, diluted volumetrically 20× with an aqueous solution containing 25% acetonitrile, and the conversion was analyzed by UPLC.

### Preparatory Example 8: Synthesis of [P(R)]-2′-deoxy-2′-fluoro-5′-O-[(R)-hydroxymercaptophosphinyl]-P-thio-β-D-arabino-adenylyl-(3′→5′)-3′-deoxy-3′-fluoroguanosine cyclic nucleotide using isolated 2′F-thio-ATP and 3′F-thio-GTP

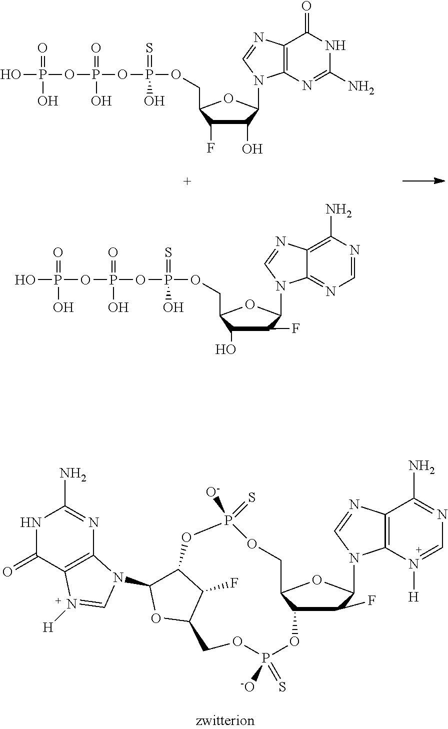

To a flask at RT under N₂ were charged 10 mL of TES (1M, 5 mmol, pH=7.5), 6 mL of 3′F-thio-GTP (0.1M, 0.3 mmol, pH=7), 1.82 mL of 2′F-thio-ATP (0.33M, 0.3 mmol, pH=7), 3 mL of CoSO₄ (0.5M, 0.75 mmol), 3 mL of ZnSO₄ (0.5M, 0.75 mmol), 10 mL of TGDE. This solution was warmed up to 35° C., and the pH was adjusted to 7.4 via 0.1N KOH solution. A wet cGAS pellet (872 mg, 15 wt % cGAS) in 8 mL of DI water was charged, and reaction mixture was aged at 35° C. for 24 h. The reaction was then quenched with NaH₂PO₄ and cooled down to RT.

### Preparatory Example 9: Synthesis of [P(R)]-2′-deoxy-2′-fluoro-5′-O-[(R)-hydroxymercaptophosphinyl]-P-thio-β-D-arabino-adenylyl-(3′→5′)-3′-deoxy-3′-fluoroguanosine cyclic nucleotide from 2′F-thio-AMP and 3′F-thio-GMP prepared using immobilized kinases

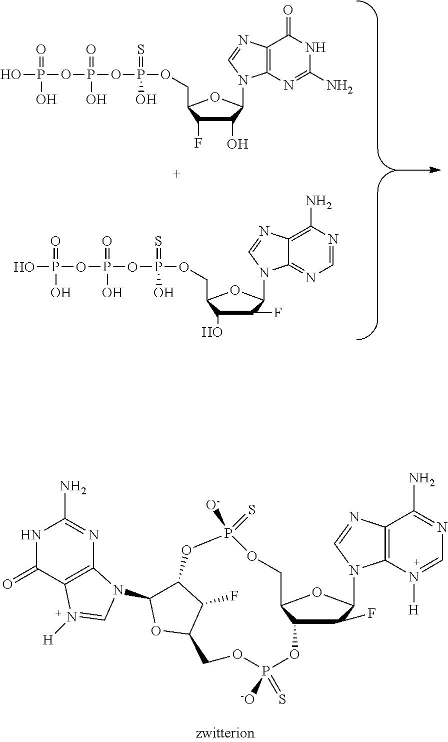

To a 100 L vessel was charged 31 L of a solution containing 2′F-thio-ATP (0.58 mol) and 3′F-thio-GTP (0.64 mol), followed by water used to rinse the container that was used to store the solution (6 L). The jacket temperature of the vessel was set to 45° C., and the agitation set to 80 RPM. N-[tris(hydroxymethyl)methyl]-2-aminoethanesulfonic acid (TES, 2.148 kg, 9.37 mol) and water used to rinse the TES container (4 L) were added, giving a pH of 6.1. The pH was then adjusted to 8.0 via addition of potassium hydroxide (0.5 L, 45 wt %). TGDE (16 L) was then added, followed by cobalt sulfate solution (1.5M, 1.1 L) and zinc sulfate solution (1.1M, 2 L), along with water used to rinse both containers (2 L). Addition of metal solutions reduced the pH to 7.4. At this time, the jacket temperature was reduced to 42° C.; the reaction temperature was 37° C. Next, cGAS enzyme slurry (8 L) was then added to initiate reaction. The reaction was aged at 35° C. for an additional 13 h until the reaction was judged to have completed (<2% 2′F-thio-ATP remaining).

### Preparatory Example 10: Synthesis of [P(R)]-2′-deoxy-2′-fluoro-5′-O-[(R)-hydroxymercaptophosphinyl]-P-thio-β-D-arabino-adenylyl-(3′→5′)-3′-deoxy-3′-fluoroguanosine cyclic nucleotide from 2′F-thio-AMP and 3′F-thio-GMP

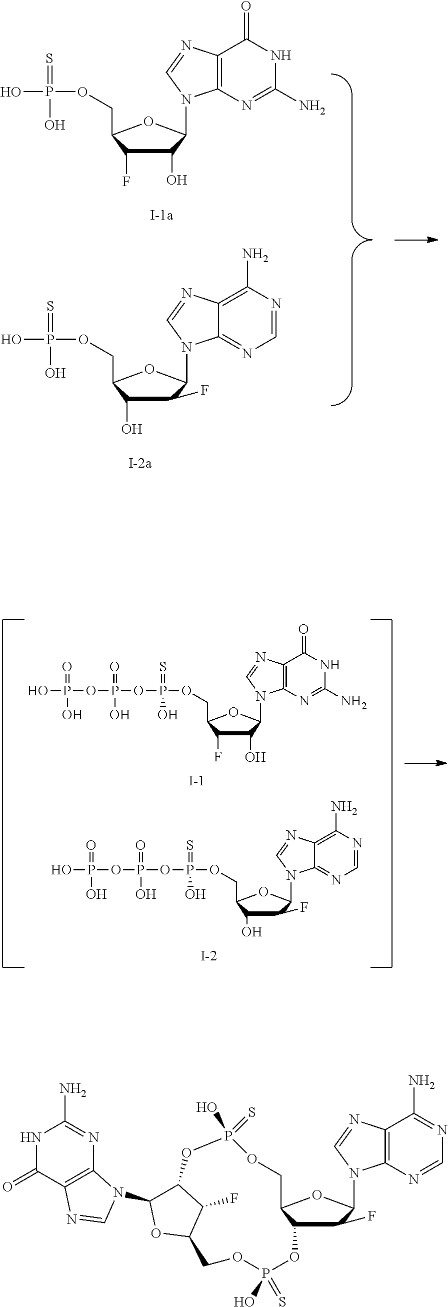

### Step 1

To a 100 L reactor was charged 2′F-thio-AMP (382.2 g, 1.0 eq) and 3′F-thio-GMP (564.7 g, 0.97 eq). The resulting mixture was then cooled down to 10° C.-15° C. followed by addition of water (33.3 L). ATP (57 mg, 0.0001 eq) was dissolved in water (60 mL) and charged to the reactor. To this, MgCl₂·6H₂O (369.2 g, 2.0 eq) was added at 10° C.-15° C., followed by addition of TES (1.041 kg, 5.0 eq). To adjust the pH of the reaction mixture from 5.20 to 5.98 (10° C.-15° C.), around 70.0 mL of KOH (45 wt %) was utilized. Next, AcP-Li/Li (752.4 g, 5.2 eq) was charged at 10° C.-12° C. Once AcP-Li/Li was fully dissolved, around 150 mL-160 mL of KOH (45 wt %) was added to adjust the pH to 7.42 at 9.5° C.-10.5° C. To this clear solution, a solution of a kinase enzyme that can be represented by SEQ ID NO: 5 (2.10 g dissolved in 0.20 L water) (as set forth above) was charged, followed by a solution of a kinase enzyme that can be represented by SEQ ID NO: 4 (2.87 g dissolved in 0.25 L water) (as set forth above) and a solution of a kinase enzyme that can be represented by SEQ ID NO: 6 (3.44 g dissolved in 0.35 L water) (as set forth above) at 9.0° C.-11° C., respectively. The reaction mixture was aged at 10° C. under nitrogen for 17 h-24 h until completion (1-3% 2′F-thio-AMP and 3′F-thio-GMP leftover).

### Step 2

Next, Na₃VO₄ (50.1 g, 0.3 eq) was charged to the reactor, followed by slow addition of a pre-cooled mixture of TGDE (15.3 L) and water (11.0 L), while maintaining the temperature below 15° C. To this, ZnSO₄·7H₂O (784.0 g, 3.0 eq) was added in one portion. Around 270 ml-285 mL of KOH (45 wt %) was charged to adjust the pH from 6.98 to 7.8 at 10° C. Then, cobalt-treated cGAS enzyme slurry that can be represented by SEQ ID NO: 7 (as set forth below) in water (22.1 kg) was charged at 10° C. Temperature was increased to 35° C., and reaction was aged at 35° C. for 15 h-24 h, until completion (<2% 3′F-thio-ATP remaining).

(SEQ ID NO: 7)
MHHHHHHGSDSEVNQEAKPEVKPEVKPETHINLKVSDGSSEIFFK
IKKTTPLRRLMEAFAKRQGKEMDSLTFLYDGIEIQADQTPEDLDM
EDNDIIEAHREQIGGENLYFQGGGPRLREVLSRLSLGRQDVSEAS
GLVNQVVSQLIQAIRSQEGSFGSIERLNTGSYYEHVKISEPNEFD
IMLVMPVSRLQLDECDDTGAFYYLTFKRNSKDKHLFKFLDEDGKL
SAFKMLQALRDIIKREVKNIKNAEVTVKRKKAGSPAITLQIKNPP
AVISVDIILTLEPQQSWPPSTQDGLKIEKWLGRKVRGQFRNKSLY
LVAKQNPREKVLRGNTWRLSFSHIEKDMLNNHGSSKTCCESDGLK
CCRKGCYKLLKYLLERLKMKYPHQLEKRSSYEVKTAFFHSCVMWP
NDLDWHLSDLDYCFQKYLGYFLDCLQKSELPHFFIPQYNLLSLED
KASNDFLSRQINYELNNRFPIFQERY

### Preparatory Example 11: Extraction Isolation of [P(R)]-2′-deoxy-2′-fluoro-5′-O-[(R)-hydroxymercaptophosphinyl]-P-thio-β-D-arabino-adenylyl-(3′→5′)-3′-deoxy-3′-fluoroguanosine cyclic nucleotide

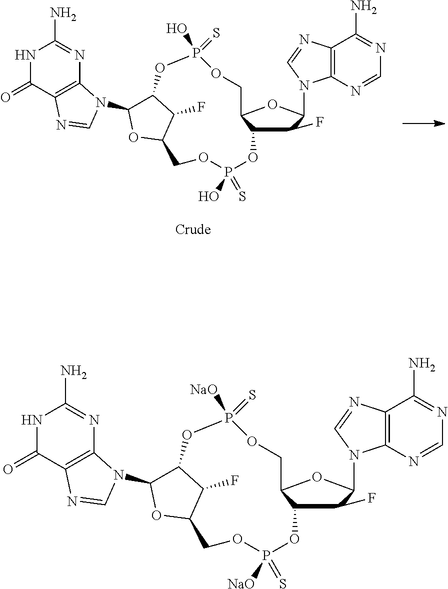

To a 100 L reactor containing 89.00 kg of the cGAS reaction mixture of Example 10 at 49.0° C. was added Na₂SO₄ (1.628 kg, 20 eq). After 2.5 h stirring at 49° C., the reactor was cooled to 10° C., and 43.88 kg of the reaction mixture was removed and stored at 0° C. To the remaining 45.12 kg of reaction mixture was added 65.4 mM [HN(n-oct)₃]₂[SO₄] in 2-Me-THF (21.9 L, 108 vol., 5.0 eq), and the reaction mixture was stirred for 25 min. The reaction mixture was cooled to −20° C., and 1-propanol (19 L, 93.3 vol.) was added. The reaction mixture was then stirred for 17 h. The reaction mixture was warmed to 50° C. and stirred for 2 h. The cell debris-rich aqueous phase was removed, and the organic phase was filtered to remove residual cell debris. The filtered organic extracts were charged into a 100 L reaction, and 0.25 wt % Na₂SO₄ in water (40 L, 196 vol.) was added. The reaction mixture was stirred for 2 h at RT. The aqueous phase was removed, and the organic phase was stored at 0° C. This step was repeated to recover additional crude product.

The organic extracts were combined in a 100 L reactor at 23° C., and water (6.6 L, 16.2 vol) was added. The mixture was stirred for 25 min. After 25 min, the aqueous phase was removed, water (6.6 L, 16.2 vol) was added, and the mixture was stirred for 25 min. After 25 min, the aqueous phase was removed, and 10% NaCl in water (4 L, 9.8 vol.) was added. The reaction mixture was stirred for 5 min, and the aqueous phase was removed.

The organic extracts were combined in a 30 L reactor at 23° C., and water (500 mL, 1.23 vol) and ION NaOH (585 mL, 1.43 vol., 10.2 eq) were added, until the mixture reached pH 13.15, over 20 min while stirring. The aqueous phase was removed, and 1N NaOH (400 mL, 0.98 vol., 0.70 eq) was added. The reaction mixture was stirred for 10 min, and the aqueous extracts were removed and combined.

The aqueous extracts were filtered through a 1p m filter and added to a 10 L reactor. The aqueous extracts were heated to 55° C. 2N HCl (400 mL, 0.98 vol., 1.40 eq) was added dropwise over 2 h to pH 7.30. The resulting slurry was cooled to 25° C. and stirred for 12 h. The product was collected by filtration and washed once with 93% EtOH: 7% water (4 L, 9.82 vol.), and again with 93% EtOH: 7% water (1.5 L, 3.68 vol.). The product was dried under air flow for 90 min then under vacuum, at a relative humidity of 32.9% to 45.0%, over 41 h.

¹H NMR (600 MHz, Deuterium Oxide) δ 8.41 (s, 1H), 8.37 (s, 1H), 8.11 (s, 1H), 6.68 (dd, J=15.0, 4.1 Hz, 1H), 6.18 (d, J=8.6 Hz, 1H), 5.90-5.66 (m, 2H), 5.58 (dd, J=53.3, 3.4 Hz, 1H), 5.41 (ddt, J=13.6, 8.3, 3.9 Hz, 1H), 4.86 (d, J=26.0 Hz, 1H), 4.61 (d, J=4.9 Hz, 1H), 4.58 (d, J=8.5 Hz, 1H), 4.31 (t, J=5.8 Hz, 2H), 4.27 (d, J=11.9 Hz, 1H).

### EXAMPLES

### Example 1: L-Histidine adduct of 2-amino-9-[(2R,5R,7R,8S,10R,12aR,14R,15S,15aR, 16R)-14-(6-amino-9H-purin-9-yl)-15,16-difluoro-2,10-dihydroxy-2,10-disulfidooctahydro-12H-5,8-methanofuro[3,2-1][1,3,6,9,11,2,10]pentaoxa-diphosphacyclotetradecin-7-yl]-1,9-dihydro-6H-purin-6-one (Form I)

Form I was produced by direct crystallization of Compound A from a saturated solution in the presence of a buffer containing L-histidine. Equilibrium solubility experiments were conducted by mixing Compound A and 50 mM L-histidine buffer (pH: 5.8<x<6.3) at a concentration of approximately 20-30 mg/mL. The sample was stirred at ambient conditions for a minimum of 16 h. At the conclusion of the solubility measurements, solutions were clarified by centrifugation. The wet precipitated material was transferred to a 96-well transmission XRPD sample plate and allowed to dry to room temperature prior to XRPD analysis.

### Example 2: Monosodium salt of 2-amino-9-[(2R,5R,7R,8S,10R,12aR,14R,15S,15aR,16R)-14-(6-amino-9H-purin-9-yl)-15,16-difluoro-2,10-dihydroxy-2,10-disulfidooctahydro-12H-5,8-methanofuro [3,2-1][1,3,6,9,11,2,10]pentaoxa-diphosphacyclotetradecin-7-yl]-1,9-dihydro-6H-purin-6-one

A monosodium salt of Compound A was produced by the following procedure. An aqueous slurry of the bis-sodium salt of Compound A was prepared in 10-15 mL of water. Dilute sodium hydroxide was added until all Compound A was dissolved. The pH of the resulting solution was approximately pH 11. Subsequently, the pH of the solution was modified with dilute hydrochloric acid to a pH range of 5-6. The mono-sodium material precipitated out to form a slurry. The slurry was stirred for up to 24 h at room temperature. The mono-sodium salt precipitate was isolated by vacuum filtration and washed with pH 5-6 water. Lastly, the material was dried under vacuum.

### Example 3: Equilibrium Solubility Studies

Equilibrium solubility experiments were conducted by mixing Compound A and the solution of interest at a concentration of approximately 20-30 mg/mL (Table 1). The solution of interest contained either 50 mM L-histidine or phosphate buffer (pH: 5.8<x<6.3). Additional Compound A was added until a cloudy solution was maintained. The samples were stirred at ambient conditions for a minimum of 16 hours. At the conclusion of the solubility measurements, solutions were clarified by centrifugation. The wet precipitated material was transferred to a 96-well transmission XRPD sample plate and allowed to dry to room temperature prior to XRPD analysis.

TABLE 1

| Compound A | | Buffer | Compound A |
|---|---|---|---|
| Starting Phase | Buffer | pH | Terminal Phase |
| Crystalline, mono- | 50 mM phosphate | 6.0 | Crystalline mono- |
| sodium salt | | | sodium salt |
| Crystalline, mono- | 50 mM L-histidine | 6.0 | Form 1 |
| sodium salt | | | |

### Example 4: X-Ray Powder Diffraction Characterization

X-ray powder diffraction (XRPD) studies are widely used to characterize molecular structures, crystallinity, and polymorphism. X-ray powder diffraction patterns for the solid phases of Compound A were generated on a Philips Analytical X'Pert PRO X-ray Diffraction System with a high throughput stage. A Cu K-Alpha radiation source was used. The diffraction peak positions were referenced by silicon (internal standard), which has a 2 theta (2θ) value of 28.4409 degree. The experiments were analyzed at ambient conditions.

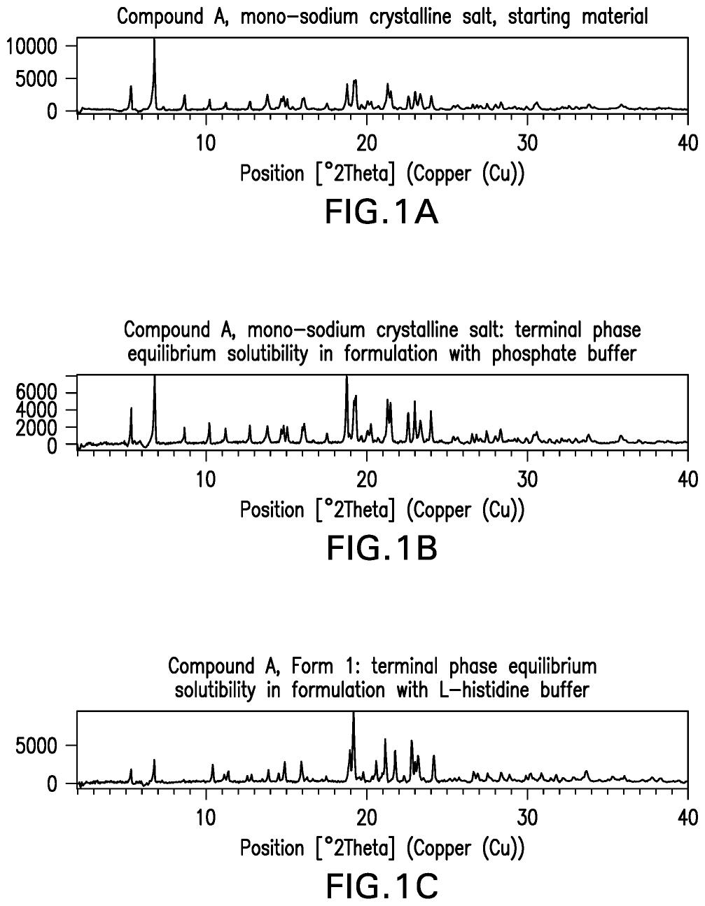

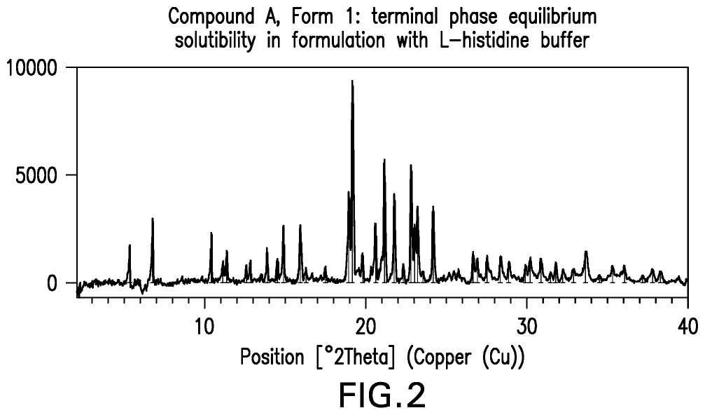

Analysis was performed on Compound A, directly as provided from synthesis as described above, and on Form I as provided in Example 1. XRPD diffraction patterns were acquired; these XRPD diffraction patterns, viewed together, compare the starting phase of Compound A, as provided from synthesis, with those phases determined at the conclusion of equilibrium solubility experiments. The phase analysis on solids isolated at the conclusion of the equilibrium solubility experiment with L-histidine buffer (FIG. 1C) is different from the starting material. The solids isolated from phosphate buffer in Example 2 (FIG. 1B), which serve as a control, maintain the phase of the Compound A starting material (FIG. 1A). The X-ray powder diffraction pattern was generated to characterize Form I, as shown in FIG. 2, which exhibited characteristic reflections, in the range of 2°-40° 2θ, corresponding to d-spacings (±0.3° 2 theta) as shown in Table 2.

TABLE 2

| Pos. [°2θ] | d-spacing [Å] | Rel. Int. [%] |
|---|---|---|
| 5.32 | 16.60 | 19 |
| 6.77 | 13.06 | 31 |
| 10.41 | 8.50 | 25 |
| 11.11 | 7.96 | 10 |
| 11.38 | 7.78 | 15 |
| 12.57 | 7.04 | 9 |
| 12.83 | 6.90 | 11 |
| 13.87 | 6.38 | 15 |
| 14.51 | 6.10 | 12 |
| 14.88 | 5.96 | 29 |
| 15.92 | 5.57 | 28 |
| 16.28 | 5.45 | 7 |
| 17.48 | 5.07 | 8 |
| 18.95 | 4.68 | 44 |
| 19.16 | 4.63 | 100 |
| 19.79 | 4.49 | 15 |
| 20.59 | 4.31 | 29 |
| 21.15 | 4.20 | 61 |
| 21.76 | 4.08 | 44 |
| 22.30 | 3.99 | 9 |
| 22.80 | 3.90 | 58 |
| 23.01 | 3.86 | 29 |
| 23.19 | 3.84 | 38 |
| 23.54 | 3.78 | 6 |
| 24.17 | 3.68 | 38 |
| 26.64 | 3.35 | 15 |
| 26.90 | 3.31 | 12 |
| 27.50 | 3.24 | 12 |
| 28.33 | 3.15 | 13 |
| 28.86 | 3.09 | 10 |
| 29.89 | 2.99 | 9 |
| 30.19 | 2.96 | 12 |
| 30.85 | 2.90 | 12 |
| 31.46 | 2.84 | 5 |
| 31.78 | 2.82 | 10 |
| 32.22 | 2.78 | 6 |
| 32.89 | 2.72 | 7 |
| 33.62 | 2.67 | 15 |
| 34.50 | 2.60 | 3 |
| 35.30 | 2.54 | 8 |
| 36.07 | 2.49 | 7 |
| 37.18 | 2.42 | 4 |
| 37.80 | 2.38 | 6 |
| 38.28 | 2.35 | 5 |

### Example 5: NMR Characterization

The composition of Form I has been characterized by ¹H NMR in solution using the following protocol. A dried cake of Form I was washed with water and dissolved either in d₆-DMSO or in D₂O, and ¹H NMR spectra were recorded with relaxation delay D1 set to 120 s for accurate quantitation. In both cases, the molar ratio of L-histidine to Compound A was 1:2.1 to 1:2.2, suggesting 1:2 stoichiometry of L-histidine to Compound A in the Form I. A representative ¹H spectrum with corresponding integrals is shown on FIG. 3.

In addition to ¹H NMR characterization of Form I composition, an interaction between Compound A and L-histidine in solution has been characterized using ¹H-NMR spectroscopy using the protocol described below.

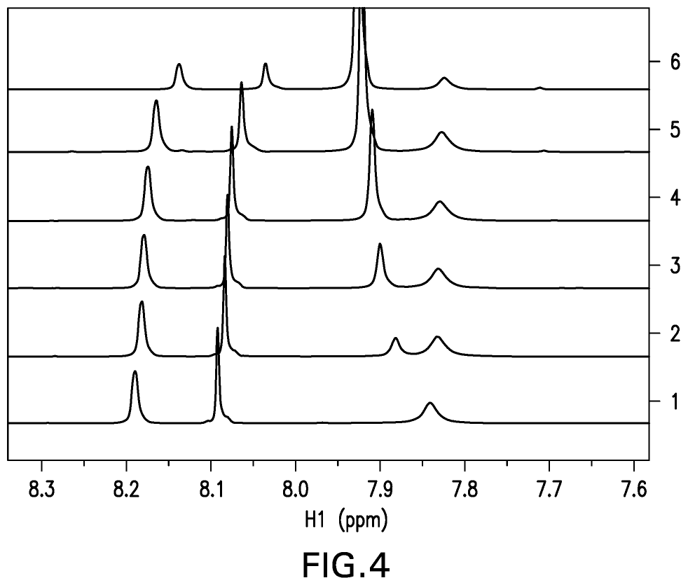

Solution ¹H-NMR studies were performed on Form I, as provided in Example 1. Six samples were prepared using with 4 mg of Compound A and 0, 0.4, 0.8, 1.6, 3.2, and 9 mg, respectively, of L-histidine in 1 mL of D₂O. Specific ¹H chemical shift changes consistent with the interaction have been observed (FIG. 4). In particular, FIG. 4 shows the aromatic region of the ¹H-NMR spectra of Compound A. Samples 1-6 contain 4 mg of Compound A and 0, 0.4, 0.8, 1.6, 3.2, and 9 mg of L-histidine, respectively, in 1 mL of D₂O. Specific chemical shift changes of aromatic ¹H nuclei of Compound A (at ˜8.1 ppm and at ˜8.2 ppm) are consistent with the binding event. These studies indicated a specific binding between L-histidine and Compound A.

It will be appreciated that various of the above-discussed and other features and functions, or alternatives thereof, may be desirably combined into many other different systems or applications. Also, that various presently unforeseen or unanticipated alternatives, modifications, variations or improvements therein may be subsequently made by those skilled in the art that are also intended to be encompassed by the following claims.

1. An adduct of 2-amino-9-[(2R,5R,7R,8S,10R,12aR,14R,15S,15aR,16R)-14-(6-amino-9H-purin-9-yl)-15,16-difluoro-2,10-dihydroxy-2,10-disulfidooctahydro-12H-5,8-methanofuro[3,2-1][1,3,6,9,11,2,10]pentaoxadiphospha-cyclotetradecin-7-yl]-1,9-dihydro-6H-purin-6-one and L-histidine (Form I).

2. The adduct of claim 1, characterized by an X-ray powder diffraction containing at least 2 of the following 2θ values measured using CuKα radiation: about 5.32, about 6.77, about 10.41, about 11.11, about 11.38, about 12.57, about 12.83, about 13.87, about 14.51, about 14.88, about 15.92, about 16.28, about 17.48, about 18.95, about 19.16, about 19.79, about 20.59, about 21.15, about 21.76, about 22.30, about 22.80, about 23.01, about 23.19, about 23.54, about 24.17, about 26.64, about 26.90, about 27.50, about 28.33, about 28.86, about 29.89, about 30.19, about 30.85, about 31.46, about 31.78, about 32.22, about 32.89, about 33.62, about 34.50, about 35.30, about 36.07, about 37.18, about 37.80, and about 38.28° 2θ.

3. The adduct of claim 2, characterized by an X-ray powder diffraction containing at least 2 of the following 2θ values measured using CuKα radiation: about 6.77, about 18.95, about 19.16, about 21.15, about 21.76, about 22.80, about 23.19, and about 24.17° 2θ.

4. The adduct of claim 2, characterized by an X-ray powder diffraction containing at least 2 of the following 2θ values measured using CuKα radiation: about 5.32, about 10.41, about 11.38, about 14.88, about 15.92, about 19.79, about 20.59, and about 23.01° 2θ.

5. The adduct of claim 2, characterized by an X-ray powder diffraction containing at least 2 of the following 2θ values measured using CuKα radiation: about 13.87, about 14.51, about 26.64, about 26.90, about 27.50, about 28.33, about 30.19, and about 33.62° 2θ.

6. The adduct of claim 2, characterized by an X-ray powder diffraction containing at least 2 of the following 2θ values measured using CuKα radiation: about 11.11, about 12.57, about 12.83, about 16.28, about 17.48, about 22.30, about 23.54, about 28.86, about 29.89, about 30.19, about 31.46, about 31.78, about 32.22, about 32.89, about 34.50, about 35.30, about 36.07, about 37.18, about 37.80, and about 38.28° 2θ.

7. The adduct of claim 1, characterized by the proton nuclear magnetic resonance (¹H-NMR) spectra of FIG. 3.

8. The adduct of claim 1, wherein the solid adduct is amorphous.

9. The adduct of claim 1, wherein the solid adduct is crystalline.

10. A pharmaceutical composition comprising an adduct of claim 1 and a pharmaceutically acceptable carrier.

11. The pharmaceutical composition of claim 10, wherein the pharmaceutical composition is a solid dosage form for oral administration.

12. The pharmaceutical composition of claim 10, wherein the pharmaceutical composition is a sterile solution for parenteral, intratumoral, intravenous, or intramuscular administration.

13. A method of inducing an immune response in a subject, comprising administering the adduct of claim 1 to the subject.

14. A method of inducing an immune response in a subject, comprising administering the pharmaceutical composition of claim 10 to the subject.

15. A method of inducing STING-dependent type 1 interferon production in a subject, comprising administering the adduct of claim 1 to the subject.

16. A method of inducing STING-dependent type 1 interferon production in a subject, comprising administering the pharmaceutical composition of claim 10 to the subject.

17. A method of treating a cell proliferation disorder in a subject, comprising administering the adduct of claim 1 to the subject.

18. The method of claim 17, wherein the cell proliferation disorder is cancer.

19. A method of treating a cell proliferation disorder in a subject, comprising administering the pharmaceutical composition of claim 10 to the subject.

20. The method of claim 19, wherein the cell proliferation disorder is cancer.


SEQUENCE LISTING (7 sequences)

SEQ ID NO: 1 - protein, 229 aa, Artificial Sequence
  Artificial ketoreductase
MHHHHHPATIVVTGGTKGIGRAIVEKFAKEGFTVLTCARTKGDNFPENVHFFKADLSKKVEVLAFADFIKQTVNQVDILVNNTGWFLPGEINNEAEGTLEAMIETNLYSAYYLTRALVGDMITKKEGHIFNICSYASIVPYTSGGSYCISKTAQLGMSKVLREELKPHHVRVTSILPGAVLNDSWAKVELPAELFIAPEDIAQIVWTAHCLPSTTVLEEILIRPQTGDL

SEQ ID NO: 2 - protein, 223 aa, Branchistoma floridae
MALPRPVVICGPSGSGKSTLYNKLLKEFPGVFQLSVSHTTRQPRPGELNGREYHFINRDQFQENIKQGDFLEWAEFSGNIYGTSKKALEEVQSNNVIPILDIDTQGVRNVKKASLEAVYIFIKPPSIDVLEKRLRSRKTETEEALQKRLSAARNELEYGLKPGNFQHIITNDDLDVAYEKLKGILIKSQMPLAMATGSSSSVVNSFLDKPAASATTVNSSSQD

SEQ ID NO: 3 - protein, 228 aa, Artificial Sequence
  adenylate kinase
MHHHHHHSSSESIRMVLIGPPGAGKGTQAPNLQERFHACHLATGDMLRSQIAKGTQLGLEAKKIMDQGGLVSDDIMVNMIKDELTNNPACKNGFILDGFPRTIPQAEKLDQMLKEQGTPLEKAVELKIDDELLPARITGRLIHPASGRSYHKIFNPPKEDMKDDVTGEALVQRSDDNADALKKRLAAYHKQTEPVVDFYKKTGIWAGVDASQPPATVWADILNKLGKD

SEQ ID NO: 4 - protein, 228 aa, Artificial Sequence
  adenylate kinase
MHHHHHHSSSESIRMVLIGPPGAGKGTQSPNLQERFHACHLGTGDMLRSQHAKGTQLGLEAKKIMDQGGLVSDDIMVNMIKDELTNNPACKNGFILDGFPRTIPQAMKLEQMLKEQGTPLEKAVELKIDDELLPARITGRLIHPASGRSYHKIFNPPKEDMKDDVTGEALVQRSDDNADALKKRLAAYHKQTEPVVDFYKKAGIWAGVDASQPVATVWADILNKLGKD

SEQ ID NO: 5 - protein, 412 aa, Artificial Sequence
  acetate kinase
MGSHHHHHGSRVLVINSGSSSIKYQLIEMEGEKVLCKGIAERIGIEGSRLVHRVGDEKHVIERELPDHEEALKLILNTLVDEKLGVIKDLKEIDAVGHRVVHGGERFKESVLVDEEVLKAIEEVSPLAPLHNPANLMGIKVAMKLLPGVPNVAVFDTAFHQTIPQKAYLYAIPYEYYEKYKIRRYGFHGTSHRYVSKRAAEILGKKLEELKIITCHIGNGASVAAVKYGKCVDTSMGFTPTEGLVMGTRSGDLDPAIPFFIMEKEGISPQEMYDILNKKSGVYGLSKGFSSDLRDIEEAALKGDEWCKLVLEIYDYRIAKYIGAYAAAMNGVDAIVFTAGVCENSPITREDVCSYLEFLGVKLDKQKNEETIDGKEGIISTPDSRVKVLVVPTNEELMIARDTKEIVEKIGR

SEQ ID NO: 6 - protein, 229 aa, Artificial Sequence
  guanylate kinase
MHHHHHHALPTPVVICGPSGSGKTTLYNKLLKEFPGVFQLTASHTTRQPRPGEENGREFHFINRDQFQENIKQGDFLEWAEHSGNLYGTSKKALEEVQANNVIPILDIDTQGVRTVKKASLEAVYIFIKPPSIEVLEERLRSRGTETEEALQKRLSAAPNELEYGLKPGNFQHIITNDDLDVAYEKLKGILIDSQMPLAGATGSSSSVVNSFLDKPAASATTVNSSSQD

SEQ ID NO: 7 - protein, 476 aa, Artificial Sequence
  cGAS
MHHHHHHGSDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGGGPRLREVLSRLSLGRQDVSEASGLVNQVVSQLIQAIRSQEGSFGSIERLNTGSYYEHVKISEPNEFDIMLVMPVSRLQLDECDDTGAFYYLTFKRNSKDKHLFKFLDEDGKLSAFKMLQALRDIIKREVKNIKNAEVTVKRKKAGSPAITLQIKNPPAVISVDIILTLEPQQSWPPSTQDGLKIEKWLGRKVRGQFRNKSLYLVAKQNPREKVLRGNTWRLSFSHIEKDMLNNHGSSKTCCESDGLKCCRKGCYKLLKYLLERLKMKYPHQLEKRSSYEVKTAFFHSCVMWPNDLDWHLSDLDYCFQKYLGYFLDCLQKSELPHFFIPQYNLLSLEDKASNDFLSRQINYELNNRFPIFQERY